(19) United States
(12) Patent Application Publication
(10) Pub. No.: US 2016/0051184 A1
(43) Pub. Date: Feb. 25, 2016

(54) SYSTEM AND METHOD FOR PROVIDING SLEEP RECOMMENDATIONS USING EARBUDS WITH BIOMETRIC SENSORS

(71) Applicant: JayBird LLC, Salt Lake City, UT (US)

(72) Inventors: BEN WISBEY, Canberra (AU); DAVID SHEPHERD, Canberra (AU); HAGEN DIESTERBECK, Little Bay (NZ); STEPHEN DUDDY, Moama (AU)

(73) Assignee: JayBird LLC, Salt Lake City, UT (US)

(21) Appl. No.: 14/933,978

(22) Filed: Nov. 5, 2015

Related U.S. Application Data
(63) Continuation-in-part of application No. 14/830,549, filed on Aug. 19, 2015.
Application No. 14/830,549 is a continuation-in-part of application No. 14/147,384, filed on Jan. 3, 2014.
Application No. 14/147,384 is a continuation-in-part of application No. 14/137,942, filed on Dec. 20, 2013.
Application No. 14/137,942 is a continuation-in-part of application No. 14/137,734, filed on Dec. 20, 2013.
Application No. 14/137,734 is a continuation-in-part of application No. 14/062,815, filed on Oct. 24, 2013.

(51) Int. Cl. A61B 5/00; A61B 5/0205

(52) U.S. Cl. CPC A61B 5/486; A61B 5/0205; A61B 5/4815; A61B 5/72; A61B 5/6817; A61B 5/7405; A61B 2562/0219; A61B 5/14552

(57) ABSTRACT

Systems and methods for providing a sleep recommendation include providing a sleep recommendation using an earphone with a biometric sensor. The system includes a preferred sleep determination module that determines a preferred sleep duration. The system also includes a sleep debt module that creates and updates a sleep debt based on the preferred sleep duration and an actual sleep duration. In addition, the system includes a sleep recommendation module that provides a recommended sleep duration based on the sleep debt.

### CROSS REFERENCE TO RELATED APPLICATIONS

This application is a continuation-in-part of and claims the benefit of U.S. patent application Ser. No. 14/830,549 filed Aug. 19, 2015, titled “Earphones with Biometric Sensors,” the contents of which are incorporated herein by reference in their entirety. This application is also a continuation-in-part of U.S. patent application Ser. No. 14/147,384, filed Jan. 3, 2014, titled “System and Method for Providing Sleep Recommendations,” which is a continuation-in-part of and claims the benefit of U.S. patent application Ser. No. 14/137,942, filed Dec. 20, 2013, titled “System and Method for Providing an Interpreted Recovery Score,” which is a continuation-in-part of U.S. patent application Ser. No. 14/137,734, filed Dec. 20, 2013, titled “System and Method for Providing a Smart Activity Score,” which is a continuation-in-part of U.S. patent application Ser. No. 14/062,815, filed Oct. 24, 2013, titled “Wristband with Removable Activity Monitoring Device.” The contents of the Ser. No. 14/830,549 application, the Ser. No. 14/147,384 application, the Ser. No. 14/137,942 application, the Ser. No. 14/137,734 application, and the Ser. No. 14/062,815 application are incorporated herein by reference in their entireties.

### TECHNICAL FIELD

The present disclosure relates generally to sleep monitoring devices, and more particularly to a system and method for providing sleep recommendations using earphones with biometric sensors.

### DESCRIPTION OF THE RELATED ART

Previous generation activity and sleep monitoring devices generally enabled only a tracking of sleep that provided an estimated sleep duration. Currently available sleep monitoring devices now add functionality that measures various parameters that may affect sleep quality. One issue is that currently available sleep monitoring devices do not learn a user's preferred sleep durations and provide sleep recommendations based on the preferred sleep durations. Another issue is that currently available solutions do not track a user's sleep debt and provide a notification that aids the user in remedying the user's sleep debt.

### BRIEF SUMMARY OF THE DISCLOSURE

In view of the above drawbacks, there exists a long-felt need for sleep monitoring devices that learn a user's preferred sleep durations and provide sleep recommendations based on the user's preferred sleep durations. Further, there is a need for sleep monitoring devices that track a user's sleep debt and provide notifications that aid the user in reducing the sleep debt and in getting to bed at a preferred bed time of the user.

Embodiments of the present disclosure include systems and methods for providing sleep recommendations.

One embodiment involves an apparatus for providing a sleep recommendation. The apparatus includes a preferred sleep determination module that determines a preferred sleep duration. The apparatus also includes a sleep debt module that creates and updates a sleep debt based on the preferred sleep duration and an actual sleep duration. In addition, the apparatus includes a sleep recommendation module that provides a recommended sleep duration based on the sleep debt.

The preferred sleep duration, in one embodiment, is based on a set of best sleep durations for a user. In a further embodiment, the set of best sleep durations is based on a set of the actual sleep durations. In one case, the apparatus includes an actual sleep determination module that determines the actual sleep duration using an accelerometer. The preferred sleep duration, in one embodiment, is based on a needed sleep duration provided by a user.

The apparatus, in another embodiment, includes a sleep reminder module that provides a sleep reminder based on the sleep debt. In one embodiment, the sleep reminder module provides the sleep reminder when the sleep debt exceeds a sleep debt threshold. The sleep reminder, in one case, includes a notification delivered to an electronic device. In one embodiment, the sleep reminder module provides the sleep reminder before a preferred bed time. In various embodiments, at least one of the preferred sleep determination module, the sleep debt module, and the sleep recommendation module is embodied in an earphone or pair of earphones with biometric sensors.

One embodiment of the present disclosure involves a method for providing a sleep recommendation. The method includes determining a preferred sleep duration. The method also includes creating and updating a sleep debt based on the preferred sleep duration and an actual sleep duration. In addition, the method includes providing a recommended sleep duration based on the sleep debt.

The preferred sleep duration, in one embodiment, is based on a set of best sleep durations for a user. In a further embodiment, the set of best sleep durations is based on a set of actual sleep durations. The actual sleep durations, in one instance, are determined using a motion sensor (e.g. an accelerometer).

In one case, the method includes providing a sleep reminder based on the sleep debt. Providing the sleep reminder, in one embodiment, occurs in response to the sleep debt exceeding a sleep debt threshold. In one case, the sleep reminder includes a notification delivered to an electronic device (e.g. a computing device such as a smartphone, smartwatch, laptop, a digital alarm clock, etc.). Providing the sleep reminder, in one embodiment, occurs before a preferred bed time. In various embodiments, at least one of the operations of determining the preferred sleep duration, creating and updating the sleep debt, and providing the recommended sleep duration includes using a sensor coupled to an earphone or pair of earphones configured to be attached to a user's body.

One embodiment of the disclosure includes a system for providing a sleep recommendation. The system includes a processor and at least one computer program residing on the processor. The computer program is stored on a non-transitory computer readable medium having computer executable program code embodied thereon. The computer executable program code is configured to determine a preferred sleep duration. The computer executable program code is also configured to create and update a sleep debt based on the preferred sleep duration and an actual sleep duration. In addition, the computer executable program code is configured to provide a recommended sleep duration based on the sleep debt.

Other features and aspects of the disclosure will become apparent from the following detailed description, taken in conjunction with the accompanying drawings, which illustrate, by way of example, the features in accordance with embodiments of the disclosure. The summary is not intended to limit the scope of the disclosure, which is defined solely by the claims attached hereto.

### BRIEF DESCRIPTION OF THE DRAWINGS

The present disclosure, in accordance with one or more various embodiments, is described in detail with reference to the following figures. The figures are provided for purposes of illustration only and merely depict typical or example embodiments of the disclosure.

FIG. 1 illustrates an example communications environment in which embodiments of the disclosed technology may be implemented.

FIG. 2A illustrates a perspective view of exemplary earphones that may be used to implement the technology disclosed herein.

FIG. 2B illustrates an example architecture for circuitry of the earphones of FIG. 2A.

FIG. 3A illustrates a perspective view of a particular embodiment of an earphone, including an optical heartrate sensor, in accordance with the disclosed technology.

FIG. 3B illustrates a side perspective view of placement of the optical heartrate sensor of the earphones of FIG. 3A when they are worn by a user.

FIG. 3C illustrates a frontal perspective view of placement of the optical heartrate sensor of the earphones of FIG. 3A when they are worn by a user.

FIG. 3D illustrates a cross-sectional view of an over-the-ear configuration of dual-fit earphones in accordance with the disclosed technology.

FIG. 3E illustrates a cross-sectional view of an over-the-ear configuration of the dual-fit earphones of FIG. 3D.

FIG. 3F illustrates a cross-sectional view of an under-the-ear configuration of the dual-fit earphones of FIG. 3D.

FIG. 4A is a block diagram illustrating an example computing device that may be used to implement embodiments of the disclosed technology.

FIG. 4B illustrates modules of an example activity monitoring application that may be used to implement embodiments of the disclosed technology.

FIG. 5 is an operational flow diagram illustrating a method of prompting a user to adjust the placement of earphones in the user's ear to ensure accurate biometric data collection by the earphones' biometric sensors.

FIG. 6 illustrates an activity display that may be associated with an activity display module of the activity monitoring application of FIG. 4B.

FIG. 7 illustrates a sleep display that may be associated with a sleep display module of the activity monitoring application of FIG. 4B.

FIG. 8 illustrates an example system for providing a sleep recommendation.

FIG. 9 illustrates an example apparatus for providing a sleep recommendation.

FIG. 10 illustrates another example apparatus for providing a sleep recommendation.

FIG. 11 is an operational flow diagram illustrating an example method for providing a sleep recommendation.

FIG. 12 is an operational flow diagram illustrating an example method for providing a sleep recommendation including providing a sleep reminder.

FIG. 13 illustrates an activity recommendation and fatigue level display that may be associated with an activity recommendation and fatigue level display module of the activity monitoring application of FIG. 4B.

FIG. 14 illustrates a biological data and intensity recommendation display that may be associated with a biological data and intensity recommendation display module of the activity monitoring application of FIG. 4B.

FIG. 15 illustrates an example computing module that may be used to implement various features of the systems and methods disclosed herein.

The figures are not intended to be exhaustive or to limit the disclosure to the precise form disclosed. It should be understood that the disclosure can be practiced with modification and alteration, and that the disclosure can be limited only by the claims and the equivalents thereof.

### DETAILED DESCRIPTION

The present disclosure is directed toward systems, methods, and apparatus for providing sleep recommendations using earphones with biometric sensors. In one such embodiment, the systems and methods are directed to an earphone or pair of earphones that provide a sleep recommendation. According to some embodiments of the disclosure, the earphone or pair of earphones are communicatively coupled to another device (e.g. a computing device such as a smartphone, smartwatch, tablet, desktop, laptop, etc.) used to provide a sleep recommendation. In one embodiment, the system includes a wearable device, and the wearable device further includes a sleep and activity monitoring device.

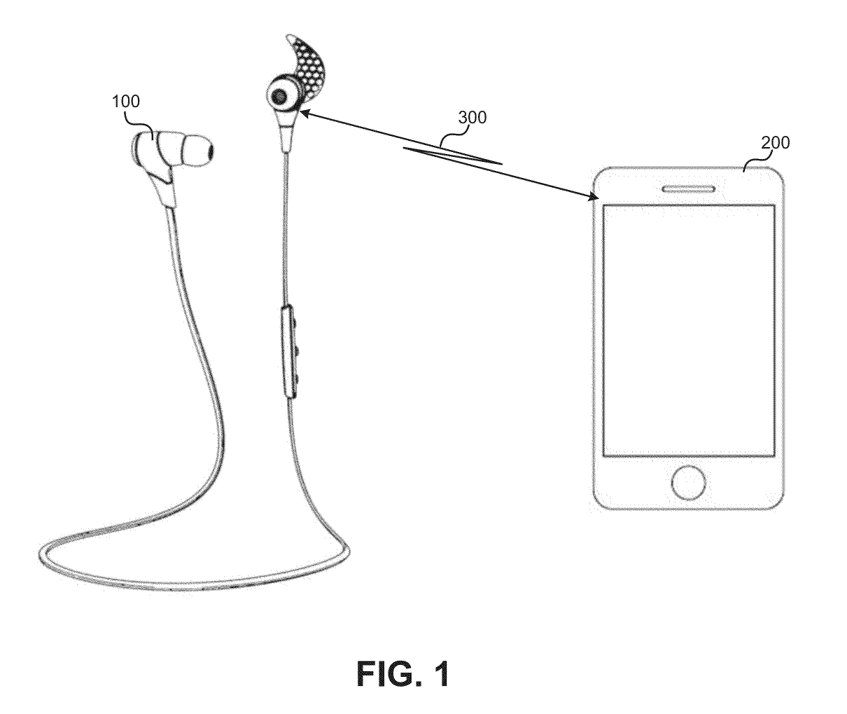

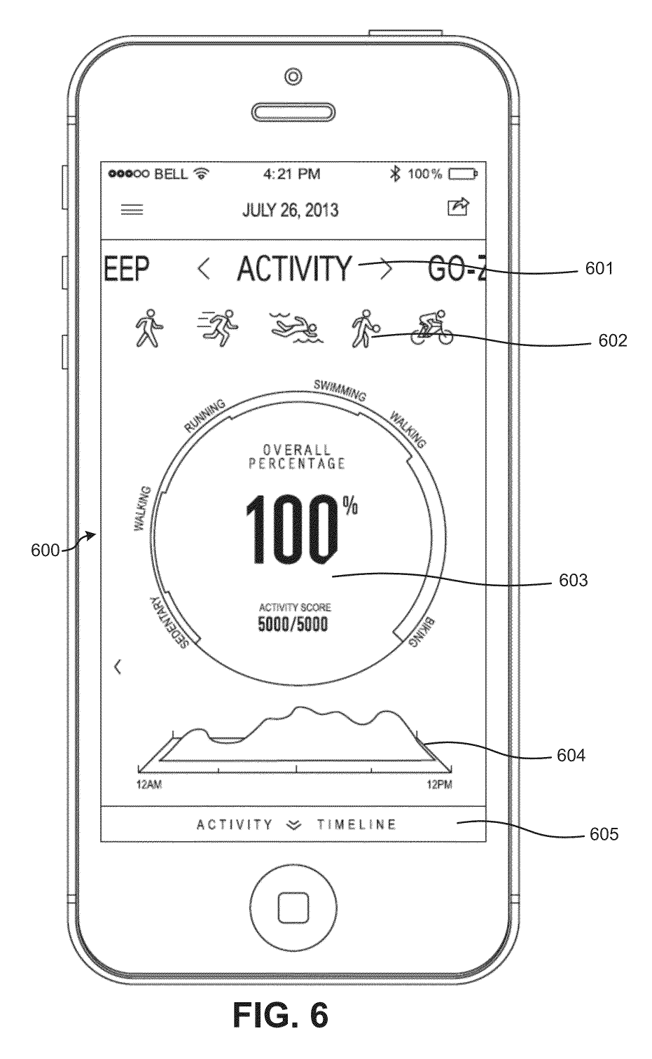

In some example implementations, one or more biometric sensors (e.g. heartrate sensor, motion sensor, etc.) are coupled to a device that is attachable to a user—for example, the attachable device may be in the form of an earphone or a pair of earphones (used interchangeably throughout this disclosure) having biometric sensors coupled thereto, and/or including an activity monitoring module. In some embodiments, such biometric earphones may be further configured with electronic components and circuitry for processing detected user biometric data and providing user biometric data to another computing device (e.g. smartphone, laptop, desktop, tablet, etc.). Because the biometric earphones of the present disclosure provide context for the disclosed systems and methods for providing sleep recommendations, various examples of the systems and methods will be described with reference to the biometric earphones as described with reference to FIGS. 1-5. Moreover, as will become clear from the disclosure with reference to FIGS. 6, 7, 13, and 14, the disclosed systems, methods, and apparatus may be implemented using any mobile or handheld device (e.g., smartphone) alone or in combination with the biometric earphones of the present disclosure.

FIG. 1 illustrates an example communications environment in accordance with an embodiment of the technology disclosed herein. In this embodiment, earphones 100 communicate biometric and audio data with computing device 200 over a communication link 300. The biometric data is measured by one or more sensors (e.g., heart rate sensor, accelerometer, gyroscope) of earphones 100. Although a smartphone is illustrated, computing device 200 may comprise any computing device (smartphone, tablet, laptop, smartwatch, desktop, etc.) configured to transmit audio data to earphones 100, receive biometric data from earphones 100 (e.g., heartrate and motion data), and process the biometric data collected by earphones 100. In additional embodiments, computing device 200 itself may collect additional biometric information that is provided for display. For example, if computing device 200 is a smartphone it may use built in accelerometers, gyroscopes, and a GPS to collect additional biometric data.

Computing device 200 additionally includes a graphical user interface (GUI) to perform functions such as accepting user input and displaying processed biometric data to the user. The GUI may be provided by various operating systems known in the art, such as, for example, iOS, Android, Windows Mobile, Windows, Mac OS, Chrome OS, Linux, Unix, a gaming platform OS, etc. The biometric information displayed to the user can include, for example a summary of the user's activities, a summary of the user's fitness levels, activity recommendations for the day, the user's heart rate and heart rate variability (HRV), and other activity related information. User input that can be accepted on the GUI can include inputs for interacting with an activity tracking application further described below.

In embodiments, the communication link 300 is a wireless communication link based on one or more wireless communication protocols such as BLUETOOTH, ZIGBEE, 602.11 protocols, Infrared (IR), Radio Frequency (RF), etc. Alternatively, the communications link 300 may be a wired link (e.g., using any one or a combination of an audio cable, a USB cable, etc.)

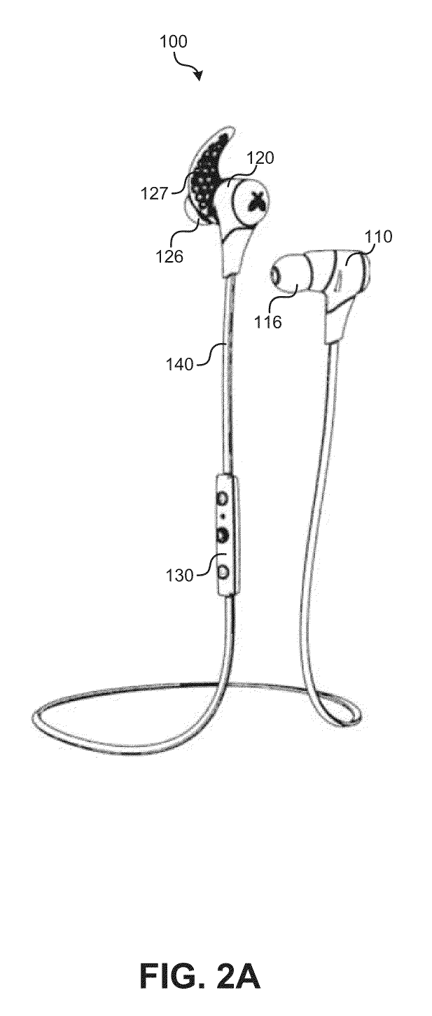

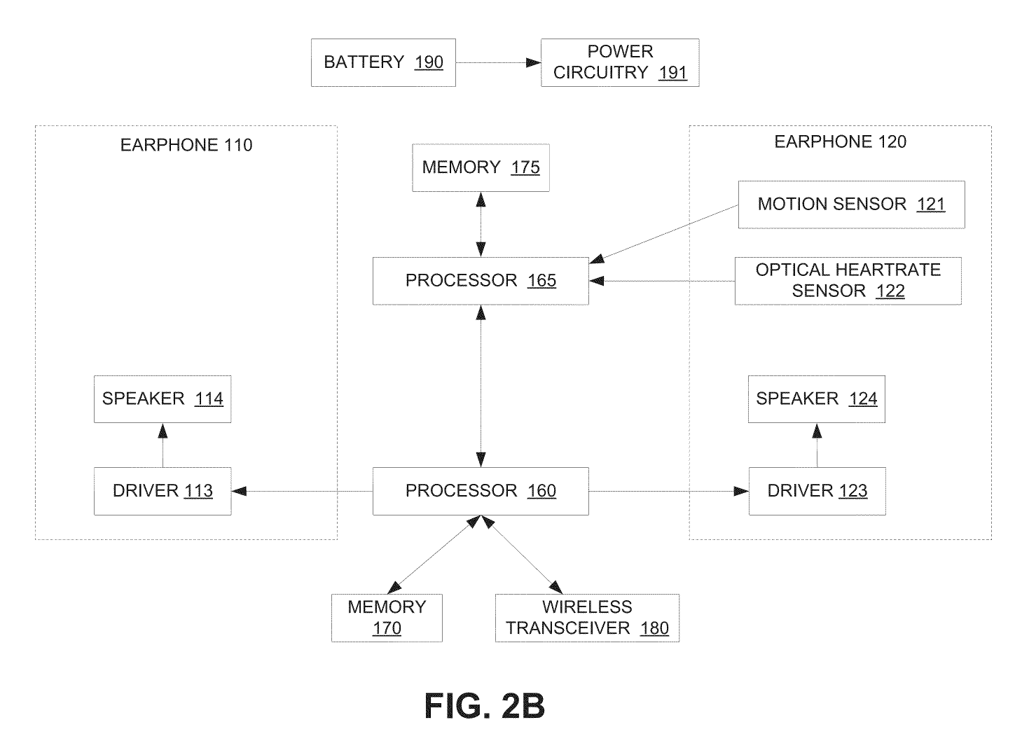

With specific reference now to earphones 100, FIG. 2A is a diagram illustrating a perspective view of exemplary earphones 100. FIG. 2A will be described in conjunction with FIG. 2B, which is a diagram illustrating an example architecture for circuitry of earphones 100. Earphones 100 comprise a left earphone 110 with tip 116, a right earphone 120 with tip 126, a controller 130 and a cable 140. Cable 140 electrically couples the left earphone 110 to the right earphone 120, and both earphones 110-120 to controller 130. Additionally, each earphone may optionally include a fin or ear cushion 127 that contacts folds in the outer ear anatomy to further secure the earphone to the wearer's ear.

In embodiments, earphones 100 may be constructed with different dimensions, including different diameters, widths, and thicknesses, in order to accommodate different human ear sizes and different preferences. In some embodiments of earphones 100, the housing of each earphone 110, 120 is rigid shell that surrounds electronic components. For example, the electronic components may include motion sensor 121, optical heartrate sensor 122, audio-electronic components such as drivers 113, 123 and speakers 114, 124, and other circuitry (e.g., processors 160, 165, and memories 170, 175). The rigid shell may be made with plastic, metal, rubber, or other materials known in the art. The housing may be cubic shaped, prism shaped, tubular shaped, cylindrical shaped, or otherwise shaped to house the electronic components.

The tips 116, 126 may be shaped to be rounded, parabolic, and/or semi-spherical, such that it comfortably and securely fits within a wearer's ear, with the distal end of the tip contacting an outer rim of the wearer's outer ear canal. In some embodiments, the tip may be removable such that it may be exchanged with alternate tips of varying dimensions, colors, or designs to accommodate a wearer's preference and/or fit more closely match the radial profile of the wearer's outer ear canal. The tip may be made with softer materials such as rubber, silicone, fabric, or other materials as would be appreciated by one of ordinary skill in the art.

In embodiments, controller 130 may provide various controls (e.g., buttons and switches) related to audio playback, such as, for example, volume adjustment, track skipping, audio track pausing, and the like. Additionally, controller 130 may include various controls related to biometric data gathering, such as, for example, controls for enabling or disabling heart rate and motion detection. In a particular embodiment, controller 130 may be a three button controller.

The circuitry of earphones 100 includes processors 160 and 165, memories 170 and 175, wireless transceiver 180, circuitry for earphone 110 and earphone 120, and a battery 190. In this embodiment, earphone 120 includes a motion sensor 121 (e.g., an accelerometer or gyroscope), an optical heartrate sensor 122, and a speaker 124 and corresponding driver 123. Earphone 110 includes a speaker 114 and corresponding driver 113. In additional embodiments, earphone 110 may also include a motion sensor (e.g., an accelerometer or gyroscope), and/or an optical heartrate sensor.

A biometric processor 165 comprises logical circuits dedicated to receiving, processing and storing biometric information collected by the biometric sensors of the earphones. More particularly, as illustrated in FIG. 2B, processor 165 is electrically coupled to motion sensor 121 and optical heartrate sensor 122, and receives and processes electrical signals generated by these sensors. These processed electrical signals represent biometric information such as the earphone wearer's motion and heartrate. Processor 165 may store the processed signals as biometric data in memory 175, which may be subsequently made available to a computing device using wireless transceiver 180. In some embodiments, sufficient memory is provided to store biometric data for transmission to a computing device for further processing.

During operation, optical heartrate sensor 122 uses a photoplethysmogram (PPG) to optically obtain the user's heart rate. In one embodiment, optical heartrate sensor 122 includes a pulse oximeter that detects blood oxygenation level changes as changes in coloration at the surface of a user's skin. More particularly, in this embodiment, the optical heartrate sensor 122 illuminates the skin of the user's ear with a light-emitting diode (LED). The light penetrates through the epidermal layers of the skin to underlying blood vessels. A portion of the light is absorbed and a portion is reflected back. The light reflected back through the skin of the user's ear is then obtained with a receiver (e.g., a photodiode) and used to determine changes in the user's blood oxygen saturation (SpO2) and pulse rate, thereby permitting calculation of the user's heart rate using algorithms known in the art (e.g., using processor 165). In this embodiment, the optical sensor may be positioned on one of the earphones such that it is proximal to the interior side of a user's tragus when the earphones are worn.

In various embodiments, optical heartrate sensor 122 may also be used to estimate a heart rate variable (HRV), i.e. the variation in time interval between consecutive heartbeats, of the user of earphones 100. For example, processor 165 may calculate the HRV using the data collected by sensor 122 based on a time domain methods, frequency domain methods, and other methods known in the art that calculate HRV based on data such as the mean heart rate, the change in pulse rate over a time interval, and other data used in the art to estimate HRV.

In further embodiments, logic circuits of processor 165 may further detect, calculate, and store metrics such as the amount of physical activity, sleep, or rest over a period of time, or the amount of time without physical activity over a period of time. The logic circuits may use the HRV, the metrics, or some combination thereof to calculate a recovery score. In various embodiments, the recovery score may indicate the user's physical condition and aptitude for further physical activity for the current day. For example, the logic circuits may detect the amount of physical activity and the amount of sleep a user experienced over the last 48 hours, combine those metrics with the user's HRV, and calculate a recovery score. In various embodiments, the calculated recovery score may be based on any scale or range, such as, for example, a range between 1 and 10, a range between 1 and 100, or a range between 0% and 100%.

During audio playback, earphones 100 wirelessly receive audio data using wireless transceiver 180. The audio data is processed by logic circuits of audio processor 160 into electrical signals that are delivered to respective drivers 113 and 123 of speaker 114 and speaker 124 of earphones 110 and 120. The electrical signals are then converted to sound using the drivers. Any driver technologies known in the art or later developed may be used. For example, moving coil drivers, electrostatic drivers, electret drivers, orthodynamic drivers, and other transducer technologies may be used to generate playback sound.

The wireless transceiver 180 is configured to communicate biometric and audio data using available wireless communications standards. For example, in some embodiments, the wireless transceiver 180 may be a BLUETOOTH transmitter, a ZIGBEE transmitter, a Wi-Fi transmitter, a GPS transmitter, a cellular transmitter, or some combination thereof. Although FIG. 2B illustrates a single wireless transceiver 180 for both transmitting biometric data and receiving audio data, in an alternative embodiment, a transmitter dedicated to transmitting only biometric data to a computing device may be used. In this alternative embodiment, the transmitter may be a low energy transmitter such as a near field communications (NFC) transmitter or a BLUETOOTH low energy (LE) transmitter. In implementations of this particular embodiment, a separate wireless receiver may be provided for receiving high fidelity audio data from an audio source. In yet additional embodiments, a wired interface (e.g., micro-USB) may be used for communicating data stored in memories 165 and 175.

FIG. 2B also shows that the electrical components of headphones 100 are powered by a battery 190 coupled to power circuitry 191. Any suitable battery or power supply technologies known in the art or later developed may be used. For example, a lithium-ion battery, aluminum-ion battery, piezo or vibration energy harvesters, photovoltaic cells, or other like devices can be used. In embodiments, battery 190 may be enclosed in earphone 110 or earphone 120. Alternatively, battery 102 may be enclosed in controller 130. In embodiments, the circuitry may be configured to enter a low-power or inactive mode when earphones 100 are not in use. For example, mechanisms such as, for example, an on/off switch, a BLUETOOTH transmission disabling button, or the like may be provided on controller 130 such that a user may manually control the on/off state of power-consuming components of earphones 100.

It should be noted that in various embodiments, processors 160 and 165, memories 170 and 175, wireless transceiver 180, motion sensor 121, optical heartrate sensor 122, and battery 190 may be enclosed in and distributed throughout any one or more of earphone 110, earphone 120, and controller 130. For example, in one particular embodiment, processor 165 and memory 175 may be enclosed in earphone 120 along with optical heartrate sensor 122 and motion sensor 121. In this particular embodiment, these four components are electrically coupled to the same printed circuit board (PCB) enclosed in earphone 120. It should also be noted that although audio processor 160 and biometric processor 165 are illustrated in this exemplary embodiment as separate processors, in an alternative embodiment the functions of the two processors may be integrated into a single processor.

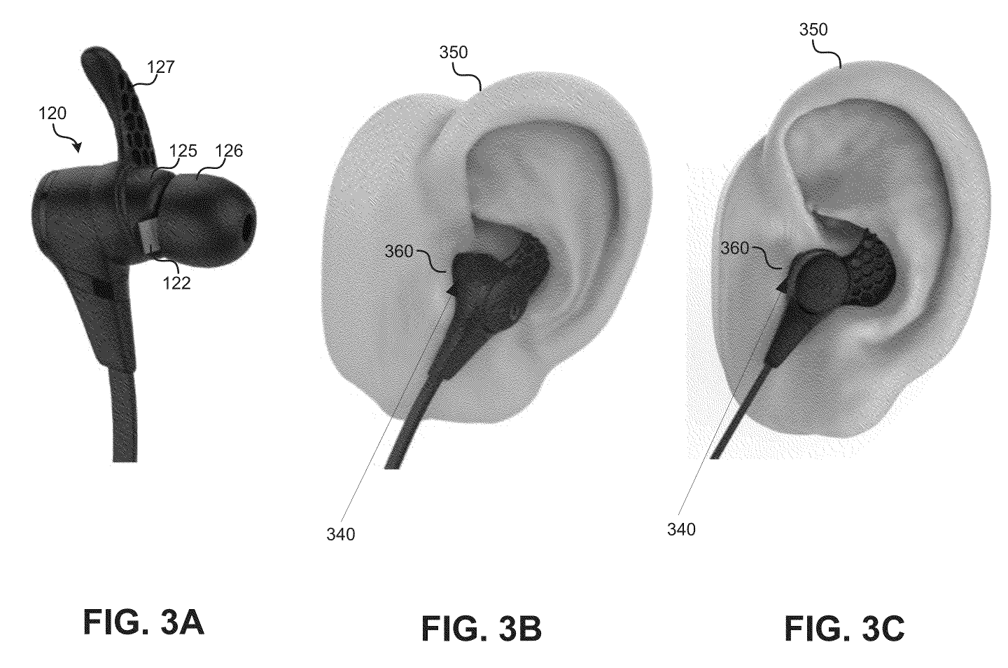

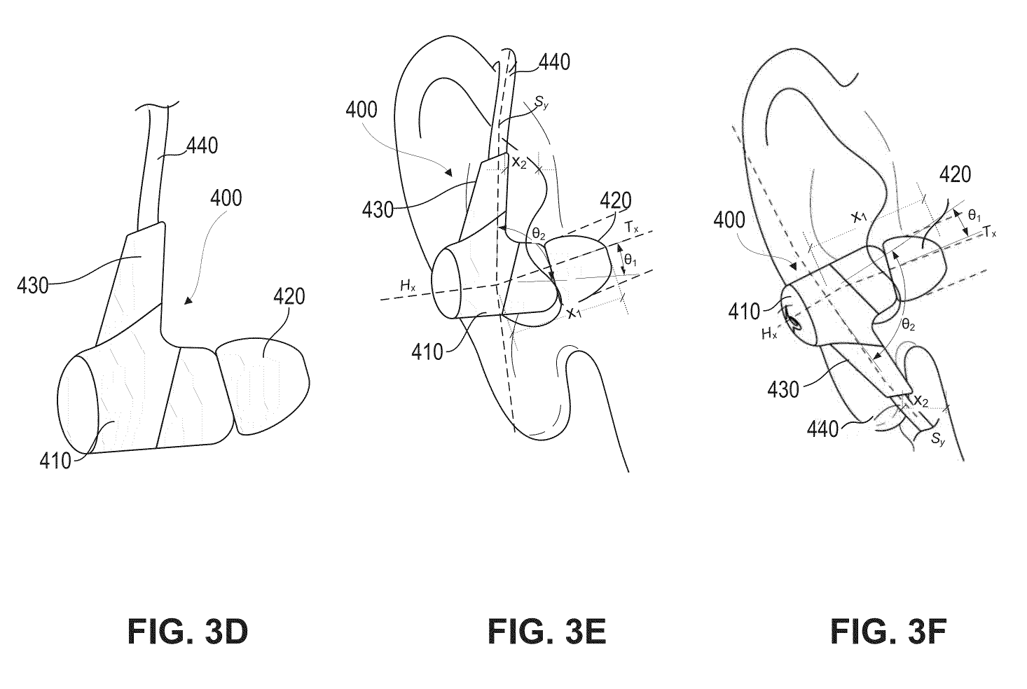

FIG. 3A illustrates a perspective view of one embodiment of an earphone 120, including an optical heartrate sensor 122, in accordance with the technology disclosed herein. FIG. 3A will be described in conjunction with FIGS. 3B-3C, which are perspective views illustrating placement of heartrate sensor 122 when earphone 120 is worn in a user's ear 350. It is important to note here that the earphone depicted in FIG. 3A is configured to be placed in a right ear of a human user, and that the earphones depicted in FIGS. 3B-3C depict the earphones being worn in a user's left ear. These alternative views are included to demonstrate that the features disclosed herein with respect to earphone 120 may be implemented in a left earphone, a right earphone, a single earphone, or both earphones. Indeed, the functionality of earphone 120 as disclosed herein, may in some embodiments be implemented in earphone 110 alone, or in combination with the same functionality implemented in earphone 120. Moreover, in some embodiments, ear cushion 127 may be removable and invertably reattached to earphone 120 such that earphone 120 may be worn in a user's left ear rather than a user's right ear. Accordingly, though the earphones in FIGS. 3B-3C will be referred to as earphone 120, the technology disclosed herein is operable whether earphone 120 is utilized as a right earphone or a left earphone.

As illustrated in FIG. 3A, earphone 120 includes a body 125, tip 126, ear cushion 127, and an optical heartrate sensor 122. Optical heartrate sensor 122 protrudes from a frontal side of body 125, proximal to tip 126 and where the earphone's nozzle (not shown) is present. FIGS. 3B-3C illustrate the optical sensor and ear interface 340 when an earphone such as earphone 120 is worn in a user's ear 350. When an earphone such as earphone 120 is worn in the ear 350 of a user, optical heartrate sensor 122 is proximal to the interior side of a user's tragus 360.

In this embodiment, optical heartrate sensor 122 illuminates the skin of the interior side of the ear's tragus 360 with a light-emitting diode (LED). The light penetrates through the epidermal layers of the skin to underlying blood vessels. A portion of the light is absorbed and a portion is reflected back. The light reflected back through the skin is then obtained with a receiver (e.g., a photodiode) of optical heartrate sensor 122 and used to determine changes in the user's blood flow, thereby permitting measurement of the user's heart rate and HRV.

In various embodiments, earphones 100 may be dual-fit earphones shaped to comfortably and securely be worn in either an over-the-ear configuration or an under-the-ear configuration. The secure fit provided by such embodiments keeps the optical heartrate sensor 122 in place on the interior side of the ear's tragus 360, thereby ensuring accurate and consistent measurements of a user's heartrate.

FIGS. 3D and 3E are cross-sectional views illustrating one such embodiment of dual-fit earphones 400 being worn in an over-the-ear configuration. FIG. 3F illustrates dual-fit earphones 400 in an under-the-ear configuration.

As illustrated, earphone 400 includes housing 410, tip 420, strain relief 430, and cord or cable 440. The proximal end of tip 420 mechanically couples to the distal end of housing 410. Similarly, the distal end of strain relief 430 mechanically couples to a side (e.g., the top side) of housing 410. Furthermore, the distal end of cord 440 is disposed within and secured by the proximal end of strain relief 430. The longitudinal axis of the housing, Hₓ, forms angle θ₁ with respect to the longitudinal axis of the tip, Tₓ. The longitudinal axis of the strain relief, S_{y}, aligns with the proximal end of strain relief 430 and forms angle θ₂ with respect to the axis Hₓ. In several embodiments, θ₁ is greater than 0 degrees (e.g., Tₓ extends in a non-straight angle from Hₓ, or in other words, the tip 420 is angled with respect to the housing 410). In some embodiments, θ₁ is selected to approximate the ear canal angle of the wearer. For example, θ₁ may range between 5 degrees and 15 degrees. Also in several embodiments, θ₂ is less than 90 degrees (e.g., S_{y}, extends in a non-orthogonal angle from Hₓ, or in other words, the strain relief 430 is angled with respect to a perpendicular orientation with housing 410). In some embodiments, θ₂ may be selected to direct the distal end of cord 440 closer to the wearer's ear. For example, θ₂ may range between 75 degrees and 85 degrees

As illustrated, x₁ represents the distance between the distal end of tip 420 and the intersection of strain relief longitudinal axis S_{y} and housing longitudinal axis Hₓ. One of skill in the art would appreciate that the dimension x₁ may be selected based on several parameters, including the desired fit to a wearer's ear based on the average human ear anatomical dimensions, the types and dimensions of electronic components (e.g., optical sensor, motion sensor, processor, memory, etc.) that must be disposed within the housing and the tip, and the specific placement of the optical sensor. In some examples, x₁ may be at least 18 mm. However, in other examples, x₁ may be smaller or greater based on the parameters discussed above.

Similarly, as illustrated, x₂ represents the distance between the proximal end of strain relief 430 and the surface wearer's ear. In the configuration illustrated, θ₂ may be selected to reduce x₂, as well as to direct the cord 440 towards the wearer's ear, such that cord 440 may rest in the crevice formed where the top of the wearer's ear meets the side of the wearer's head. In some embodiments, θ₂ may range between 75 degrees and 85 degrees. In some examples, strain relief 430 may be made of a flexible material such as rubber, silicone, or soft plastic such that it may be further bent towards the wearer's ear. Similarly, strain relief 430 may comprise a shape memory material such that it may be bent inward and retain the shape. In some examples, strain relief 630 may be shaped to curve inward towards the wearer's ear.

In some embodiments, the proximal end of tip 420 may flexibly couple to the distal end of housing 410, enabling a wearer to adjust θ₁ to most closely accommodate the fit of tip 420 into the wearer's ear canal (e.g., by closely matching the ear canal angle).

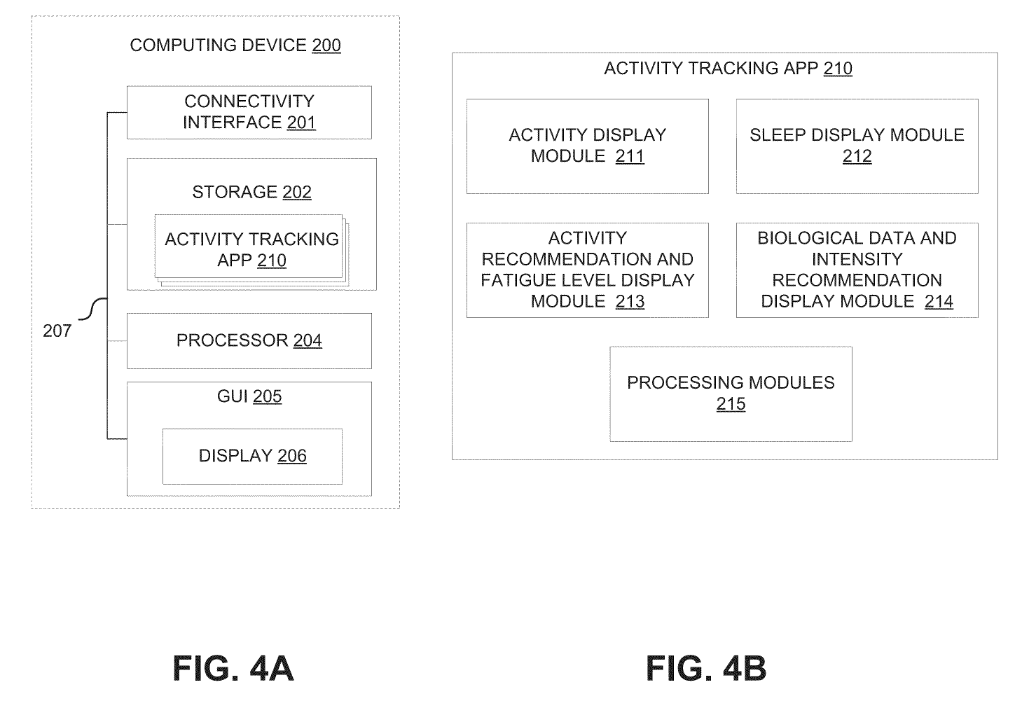

As one having skill in the art would appreciate from the above description, earphones 100 in various embodiments may gather biometric user data that may be used to track a user's activities and activity level. That data may then be made available to a computing device, which may provide a GUI for interacting with the data using a software activity tracking application installed on the computing device. FIG. 4A is a block diagram illustrating example components of one such computing device 200 including an installed activity tracking application 210.

As illustrated in this example, computing device 200 comprises a connectivity interface 201, storage 202 with activity tracking application 210, processor 204, a graphical user interface (GUI) 205 including display 206, and a bus 207 for transferring data between the various components of computing device 200.

Connectivity interface 201 connects computing device 200 to earphones 100 through a communication medium. The medium may comprise a wireless network system such as a BLUETOOTH system, a ZIGBEE system, an Infrared (IR) system, a Radio Frequency (RF) system, a cellular network, a satellite network, a wireless local area network, or the like. The medium may additionally comprise a wired component such as a USB system.

Storage 202 may comprise volatile memory (e.g. RAM), non-volatile memory (e.g. flash storage), or some combination thereof. In various embodiments, storage 202 may store biometric data collected by earphones 100. Additionally, storage 202 stores an activity tracking application 210, that when executed by processor 204, allows a user to interact with the collected biometric information.

In various embodiments, a user may interact with activity tracking application 210 via a GUI 205 including a display 206, such as, for example, a touchscreen display that accepts various hand gestures as inputs. In accordance with various embodiments, activity tracking application 210 may process the biometric information collected by earphones 100 and present it via display 206 of GUI 205. Before describing activity tracking application 210 in further detail, it is worth noting that in some embodiments earphones 100 may filter the collected biometric information prior to transmitting the biometric information to computing device 200. Accordingly, although the embodiments disclosed herein are described with reference to activity tracking application 210 processing the received biometric information, in various implementations various preprocessing operations may be performed by a processor 160, 165 of earphones 100.

In various embodiments, activity tracking application 210 may be initially configured/setup (e.g., after installation on a smartphone) based on a user's self-reported biological information, sleep information, and activity preference information. For example, during setup a user may be prompted via display 206 for biological information such as the user's gender, height, age, and weight. Further, during setup the user may be prompted for sleep information such as the amount of sleep needed by the user and the user's regular bed time. Further, still, the user may be prompted during setup for a preferred activity level and activities the user desires to be tracked (e.g., running, walking, swimming, biking, etc.) In various embodiments, described below, this self-reported information may be used in tandem with the information collected by earphones 100 to display activity monitoring information using various modules.

Following setup, activity tracking application 210 may be used by a user to monitor and define how active the user wants to be on a day-to-day basis based on the biometric information (e.g., accelerometer information, optical heart rate sensor information, etc.) collected by earphones 100. As illustrated in FIG. 4B, activity tracking application 210 may comprise various display modules, including an activity display module 211, a sleep display module 212, an activity recommendation and fatigue level display module 213, and a biological data and intensity recommendation display module 214. Additionally, activity tracking application 210 may comprise various processing modules 215 for processing the activity monitoring information (e.g., optical heartrate information, accelerometer information, gyroscope information, etc.) collected by the earphones or the biological information entered by the users. These modules may be implemented separately or in combination. For example, in some embodiments activity processing modules 215 may be directly integrated with one or more of display modules 211-214.

As will be further described below, each of display modules 211-214 may be associated with a unique display provided by activity tracking app 210 via display 206. That is, in some embodiments, activity display module 211 may have an associated activity display, sleep display module 212 may have an associated sleep display, activity recommendation and fatigue level display module 213 may have an associated activity recommendation and fatigue level display, and biological data and intensity recommendation display module 214 may have an associated biological data and intensity recommendation display.

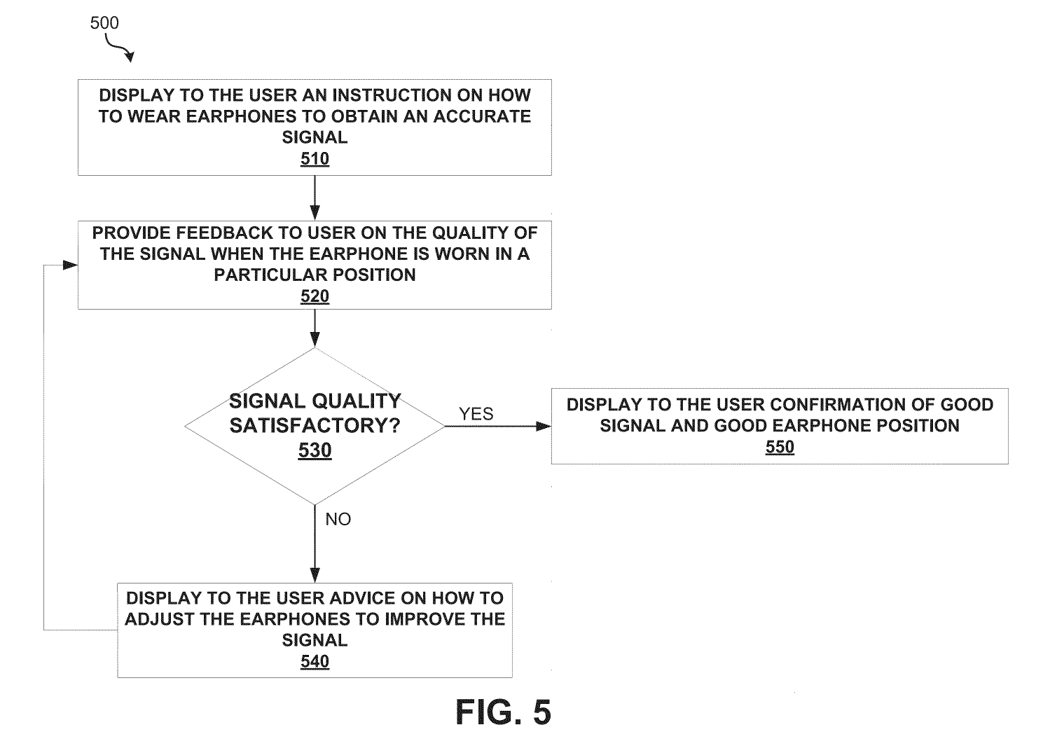

In embodiments, application 210 may be used to display to the user an instruction for wearing and/or adjusting earphones 100 if it is determined that optical heartrate sensor 122 and/or motion sensor 121 are not accurately gathering motion data and heart rate data. FIG. 5 is an operational flow diagram illustrating one such method 500 of an earphone adjustment feedback loop with a user that ensures accurate biometric data collection by earphones 100. At operation 510, execution of application 210 may cause display 206 to display an instruction to the user on how to wear earphones 100 to obtain an accurate and reliable signal from the biometric sensors. In embodiments, operation 510 may occur once after installing application 210, once a day (e.g., when user first wears the earphones 100 for the day), or at any custom and/or predetermined interval.

At operation 520, feedback is displayed to the user regarding the quality of the signal received from the biometric sensors based on the particular position that earphones 100 are being worn. For example, display 206 may display a signal quality bar or other graphical element. At decision 530, it is determined if the biosensor signal quality is satisfactory for biometric data gathering and use of application 210. In various embodiments, this determination may be based on factors such as, for example, the frequency with which optical heartrate sensor 122 is collecting heart rate data, the variance in the measurements of optical heartrate sensor 122, dropouts in heart rate measurements by sensor 122, the signal-to-noise ratio approximation of optical heartrate sensor 122, the amplitude of the signals generated by the sensors, and the like.

If the signal quality is unsatisfactory, at operation 540, application 210 may cause display 206 to display to the user advice on how to adjust the earphones to improve the signal, and operations 520 and decision 530 may subsequently be repeated. For example, advice on adjusting the strain relief of the earphones may be displayed. Otherwise, if the signal quality is satisfactory, at operation 550, application may cause display 206 to display to the user confirmation of good signal quality and/or good earphone position. Subsequently, application 210 may proceed with normal operation (e.g., display modules 211-214). FIGS. 6, 7, 13, and 14 illustrate anexemplary implementation of a GUI for app 210 comprising displays associated with each of display modules 211-214.

FIG. 6 illustrates an activity display 600 that may be associated with an activity display module 211. In various embodiments, activity display 600 may visually present to a user a record of the user's activity. As illustrated, activity display 600 may comprise a display navigation area 601, activity icons 602, activity goal section 603, live activity chart 604, and activity timeline 605. As illustrated in this particular embodiment, display navigation area 601 allows a user to navigate between the various displays associated with modules 211-214 by selecting “right” and “left” arrows depicted at the top of the display on either side of the display screen title. An identification of the selected display may be displayed at the center of the navigation area 601. Other selectable displays may displayed on the left and right sides of navigation area 601. For example, in this embodiment the activity display 600 includes the identification “ACTIVITY” at the center of the navigation area. If the user wishes to navigate to a sleep display in this embodiment, the user may select the left arrow. In implementations where device 200 includes a touch screen display, navigation between the displays may be accomplished via finger swiping gestures. For example, in one embodiment a user may swipe the screen right or left to navigate to a different display screen. In another embodiment, a user may press the left or right arrows to navigate between the various display screens.

In various embodiments, activity icons 602 may be displayed on activity display 600 based on the user's predicted or self-reported activity. For example, in this particular embodiment activity icons 602 are displayed for the activities of walking, running, swimming, sport, and biking, indicating that the user has performed these five activities. In one particular embodiment, one or more modules of application 210 may estimate the activity being performed (e.g., sleeping, walking, running, or swimming) by comparing the data collected by a biometric earphone's sensors to pre-loaded or learned activity profiles. For example, accelerometer data, gyroscope data, heartrate data, or some combination thereof may be compared to preloaded activity profiles of what the data should look like for a generic user that is running, walking, or swimming. In implementations of this embodiment, the preloaded activity profiles for each particular activity (e.g., sleeping, running, walking, or swimming) may be adjusted over time based on a history of the user's activity, thereby improving the activity predictive capability of the system. In additional implementations, activity display 600 allows a user to manually select the activity being performed (e.g., via touch gestures), thereby enabling the system to accurately adjust an activity profile associated with the user-selected activity. In this way, the system's activity estimating capabilities will improve over time as the system learns how particular activity profiles match an individual user. Particular methods of implementing this activity estimation and activity profile learning capability are described in U.S. patent application Ser. No. 14/568,835, filed Dec. 12, 2014, titled “System and Method for Creating a Dynamic Activity Profile”, and which is incorporated herein by reference in its entirety.

In various embodiments, an activity goal section 603 may display various activity metrics such as a percentage activity goal providing an overview of the status of an activity goal for a timeframe (e.g., day or week), an activity score or other smart activity score associated with the goal, and activities for the measured timeframe (e.g., day or week). For example, the display may provide a user with a current activity score for the day versus a target activity score for the day. Particular methods of calculating activity scores are described in U.S. patent application Ser. No. 14/137,734, filed Dec. 20, 2013, titled “System and Method for Providing a Smart Activity Score”, and which is incorporated herein by reference in its entirety.

In various embodiments, the percentage activity goal may be selected by the user (e.g., by a touch tap) to display to the user an amount of a particular activity (e.g., walking or running) needed to complete the activity goal (e.g., reach 100%). In additional embodiments, activities for the timeframe may be individually selected to display metrics of the selected activity such as points, calories, duration, or some combination thereof. For example, in this particular embodiment activity goal section 603 displays that 100% of the activity goal for the day has been accomplished. Further, activity goal section 603 displays that activities of walking, running, biking, and no activity (sedentary) were performed during the day. This is also displayed as a numerical activity score 5000/5000. In this embodiment, a breakdown of metrics for each activity (e.g., activity points, calories, and duration) for the day may be displayed by selecting the activity.

A live activity chart 604 may also display an activity trend of the aforementioned metrics (or other metrics) as a dynamic graph at the bottom of the display. For example, the graph may be used to show when user has been most active during the day (e.g., burning the most calories or otherwise engaged in an activity).

An activity timeline 605 may be displayed as a collapsed bar at the bottom of display 600. In various embodiments, when a user selects activity timeline 605, it may display a more detailed breakdown of daily activity, including, for example, an activity performed at a particular time with associated metrics, total active time for the measuring period, total inactive time for the measuring period, total calories burned for the measuring period, total distance traversed for the measuring period, and other metrics.

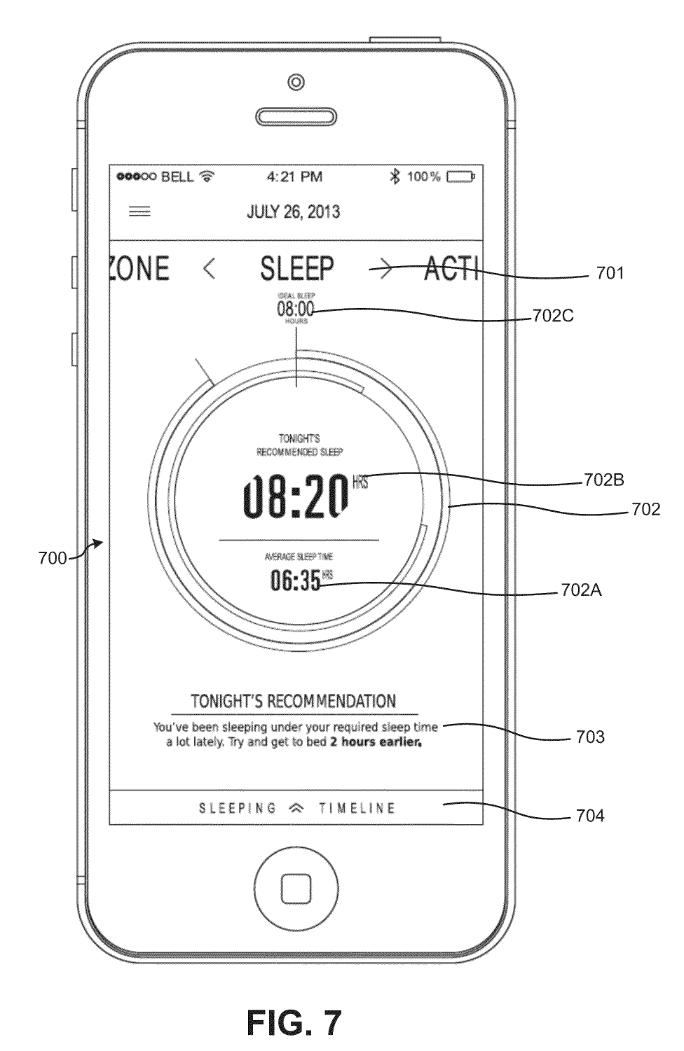

FIG. 7 illustrates a sleep display 700 that may be associated with a sleep display module 212. In various embodiments, sleep display 700 may visually present to a user a record of the user's sleep history and sleep recommendations for the day. It is worth noting that in various embodiments one or more modules of the activity tracking application 210 may automatically determine or estimate when a user is sleeping (and awake) based on an a pre-loaded or learned activity profile for sleep, in accordance with the activity profiles described above. Alternatively, the user may interact with the sleep display 700 or other display to indicate that the current activity is sleep, enabling the system to better learn that individualized activity profile associated with sleep. The modules may also use data collected from the earphones, including fatigue level and activity score trends, to calculate a recommended amount of sleep. Systems and methods for implementing this functionality are described in greater detail in U.S. patent application Ser. No. 14/568,835, filed Dec. 12, 2014, and titled “System and Method for Creating a Dynamic Activity Profile”, U.S. patent application Ser. No. 14/137,942, filed Dec. 20, 2013, titled “System and Method for Providing an Interpreted Recovery Score,” and U.S. patent application Ser. No. 14/147,384, filed Jan. 3, 2014, titled “System and Method of Providing Sleep Recommendations,” each of which is incorporated herein by reference in their entirety.

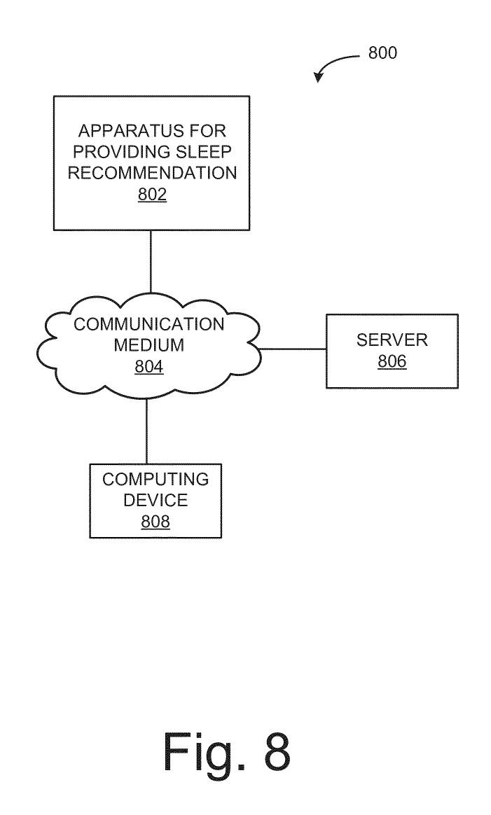

For example, FIG. 8 is a schematic block diagram illustrating example system 800 for providing a sleep recommendation. System 800 includes apparatus for providing a sleep recommendation 802, communication medium 804, server 806, and computing device 808.

Communication medium 804 may be implemented in a variety of forms. For example, communication medium 804 may be an Internet connection, such as a local area network (“LAN”), a wide area network (“WAN”), a fiber optic network, internet over power lines, a hard-wired connection (e.g., a bus), and the like, or any other kind of network connection or series of network connections. Communication medium 804 may be implemented using any combination of routers, cables, modems, switches, fiber optics, wires, radio, and the like. Communication medium 804 may be implemented using various wireless standards, such as Bluetooth, Wi-Fi, 4G LTE, etc. One of skill in the art will recognize other ways to implement communication medium 804 to establish, for example, a communication link 300 as illustrated in FIG. 1, for communications purposes.

Server 806 directs communications made over communication medium 804. Server 806 may be, for example, an Internet server, a router, a desktop or laptop computer, a smartphone, a tablet, a processor, a module, or the like. In one embodiment, server 806 directs communications between communication medium 804 and computing device 808. For example, server 806 may update information stored on computing device 808, or server 806 may send information to computing device 808 in real time.

Computing device 808 may take a variety of forms, such as a desktop or laptop computer, a smartphone, a tablet, a processor, a module, or the like. In some embodiments, computing device 708 includes computing device 200 depicted in FIG. 4A. In other embodiments, computing device 708 may be embodied in earphones 100 of FIGS. 1-2. In addition, computing device 808 may be a processor or module embedded in a wearable sensor (e.g. biometric earphones 100), a bracelet, a smart-watch, a piece of clothing, an accessory, and so on. For example, computing device 808 may also be, for example, substantially similar to devices embedded in biometric earphones 100, as illustrated in FIGS. 1-3F. Computing device 808 may communicate with other devices over communication medium 804 with or without the use of server 806. In one embodiment, computing device 808 includes apparatus 802. In various embodiments, apparatus 802 may be used to perform various processes described herein, and/or may be used to execute various operations described herein with regard to one or more disclosed systems and methods. For example, computer program code stored on one or more of biometric earphone processors 160, 165 may, when executed, perform any one or more of the operations performed by any one or more of the modules described in more detail below.

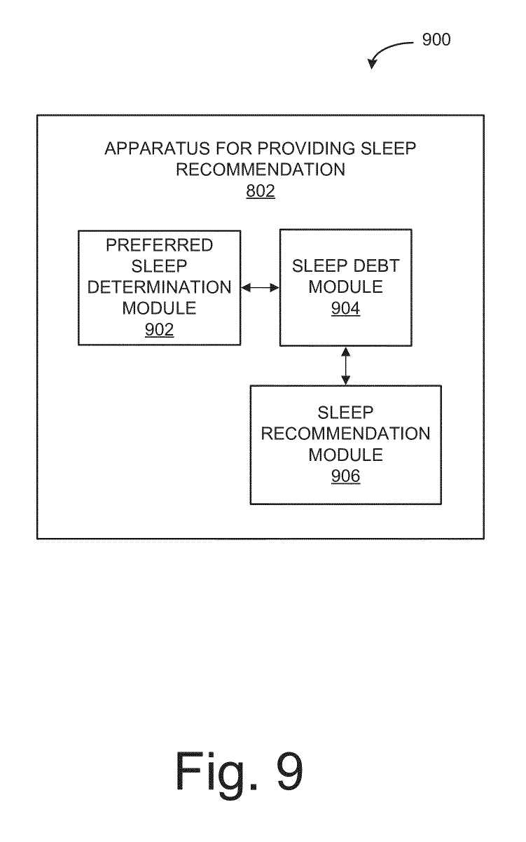

FIG. 9 is a schematic block diagram illustrating one embodiment of apparatus for providing a sleep recommendation 900. Apparatus 900 includes apparatus 802 with preferred sleep determination module 902, sleep debt module 904, and sleep recommendation module 906.

Preferred sleep determination module 902 determines a preferred sleep duration. Preferred sleep determination module 902 will be described below in further detail with regard to various processes.

Sleep debt module 904 creates and updates a sleep debt based on the preferred sleep duration and an actual sleep duration. Sleep debt module 904 will be described below in further detail with regard to various processes.

Sleep recommendation module 906 provides a recommended sleep duration based on the sleep debt. Sleep recommendation module 906 will be described below in further detail with regard to various processes.

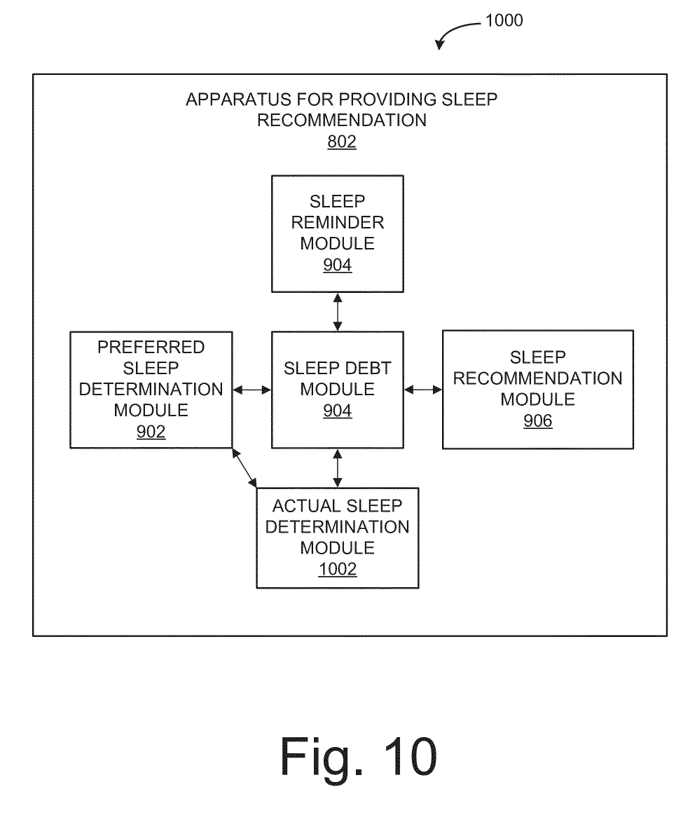

FIG. 10 is a schematic block diagram illustrating one embodiment of apparatus for providing a sleep recommendation 1000. Apparatus 1000 includes apparatus 802 with preferred sleep determination module 902, sleep debt module 904, and sleep recommendation module 906. Apparatus 1000 also includes actual sleep determination module 1002 that determines the actual sleep duration using information obtained from a motion sensor (e.g. an accelerometer). In addition, apparatus 1000 includes sleep reminder module 904 that provides a sleep reminder based on the sleep debt. Actual sleep determination module 1002 and sleep reminder module 904 will be described below in further detail with regard to various processes.

In one embodiment, at least one of preferred sleep determination module 902, sleep debt module 904, sleep recommendation module 906, actual sleep determination module 1002, and sleep reminder module 904 is embodied in a wearable sensor, such as biometric earphones 100. In various embodiments, any one or more of the modules described herein are embodied in biometric earphones 100 and connect to other modules described herein via communication medium 804. In some embodiments, one or more of the modules described herein are embodied in computing device 808 (e.g. computing device 200) and connect to other modules embodied in apparatus 802 (e.g. biometric earphones 100) described herein via communication medium 804 (e.g. over communications link 300). The computing device 808 may further be configured with additional sensors that may, in combination with the sensors of the biometric earphones, provide enhanced precision and accuracy.

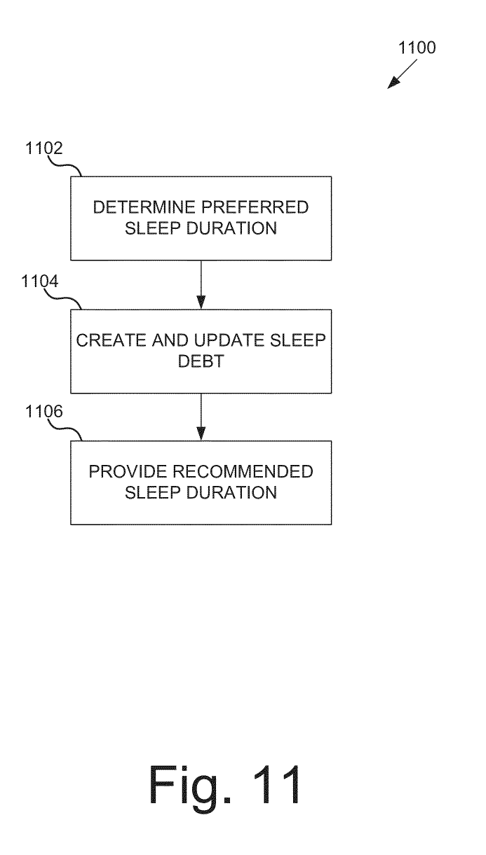

FIG. 11 is an operational flow diagram illustrating example method 1100 for providing a sleep recommendation in accordance with an embodiment of the present disclosure. The operations of method 1100 provide a sleep recommendation that is tuned specifically to a user's measured preferred sleep durations, as well as to the user's sleep debt. This aids in providing sleep recommendations that are specifically tailored to the user and that help the user eliminate sleep debt and get back on track with the user's preferred sleep patterns. In one embodiment, apparatus 802, earphones 100, and computing device 808 perform various operations of method 1100.

At operation 1102, method 1100 involves determining a preferred sleep duration. The preferred sleep duration, in one embodiment, includes an amount of time, measured in hours and minutes, etc. In one embodiment of method 1100, an estimated preferred sleep duration—or needed sleep duration—is provided by a user as an initial matter. The user may enter the user's estimated preferred sleep duration via a user interface (e.g. GUI 205, controller 130, etc.). As users typically are not able to provide accurate predictions for the estimated preferred sleep duration, the estimated preferred sleep duration may serve as a rough baseline in determining the user's preferred sleep duration.

In one embodiment, as more sleep data is gathered—i.e., as the user's actual sleep durations are measured—the estimated preferred sleep duration is phased out and replaced by an empirical preferred sleep duration. Whereas the estimated preferred sleep duration is based on an estimate provided by the user, the empirical preferred sleep duration is based on measured actual sleep duration. The actual sleep duration, in one embodiment, is determined using a motion sensor (e.g. an accelerometer). In another embodiment, the actual sleep duration is determined using an optical heartrate sensor that detects when a user's heartrate falls within a range of heartrates that correspond to the heartrate of the user when the user is sleeping. In another embodiment, the actual sleep duration may similarly be determined using an optical heartrate sensor to determine HRV. In a further, embodiment, the actual sleep determination is further determined based on input from the user. For example, the user may indicate that the user is going to bed, at which point the motion sensor (e.g. an accelerometer) or heartrate sensor (e.g. optical heartrate sensor) may begin to detect whether or not the user is asleep. The preferred sleep duration, in one illustrative example, includes a weighted combination of the estimated preferred sleep duration and the empirical preferred sleep duration. As more data is gathered, the empirical preferred sleep duration may be weighted more heavily and the estimated preferred sleep duration weighted less heavily.

For example, when the user initially provides the estimated preferred sleep duration, the estimated preferred sleep duration may be weighted to 100%. If the estimated preferred sleep duration is 8.0 hours, then the preferred sleep duration may be determined to be 8.0 hours. Then, after one week of measuring the user's actual sleep durations, the empirical preferred sleep duration may be 7.0 hours. If, for example, the weighting after one week were 50/50, the preferred sleep duration may be determined to be 7.5 hours.

After gathering a substantial amount of actual sleep data, the empirical preferred sleep duration may likely be more reliable than the estimated preferred sleep duration, and thus may eventually be weighted 100%, with the estimated preferred sleep duration weighted 0%. In other words, in this embodiment, the empirical preferred sleep duration gradually phases out the estimated sleep duration as the user's true (measured) preferred sleep duration is learned. The rate at which the empirical preferred sleep duration phases out the estimated sleep duration may depend on various factors. For example, the rate my depend on the difference between the empirical preferred sleep duration and the estimated sleep duration, the rate of change of the preferred sleep duration, and the like.

The preferred sleep duration, in one embodiment, is substantially based on the empirical preferred sleep duration. In one instance, the empirical preferred sleep duration is based on a set of best sleep durations for the user. The set of best sleep durations, in one embodiment, is based on a set of the actual sleep durations. The best sleep durations, by way of example, may include a set of the user's longest actual sleep durations (i.e., the best sleep duration may be a subset of the actual sleep durations). In such an example, the preferred sleep duration may be the mean of the user's best sleep durations. To illustrate, if thirty actual sleep durations have been measured, the best sleep durations may include the top two-thirds longest actual sleep durations. In such an example, the preferred sleep duration would be averaged only from those top two-thirds longest actual sleep durations (i.e., the best sleep durations), and the bottom one thirds, representing the shortest actual sleep durations, would not factor in to the preferred sleep duration.

In one embodiment, method 1100 involves detecting causes of the user's best sleep (or best sleep causes). For example, the user might achieve the user's best sleep when the user exercises in the morning, or when the user refrains from drinking Diet Coke®. In such examples, these causes for the best sleep are detected and presented to the user. This may aid the user in attaining the user's best sleep and in eliminating sleep debt. The best sleep causes, in one embodiment, are detected automatically by detecting patterns of activities that precede the user's best sleep durations. By way of example, method 1100 may detect that in 90% of the user's best sleep durations, the user exercised in the morning before the best sleep duration. In another embodiment, the user is prompted following the best sleep duration as to what the user thinks was the cause of the best sleep. This may be done through a user interface.

After detecting the best sleep causes, in one embodiment, the user is provided with suggestions to aid in achieving the best sleep duration. Such suggestions may include the best sleep causes. For example, if morning exercise is detected as a best sleep cause for the user, the user may receive a suggestion that the user should exercise in the morning. The best sleep cause suggestions may be provided by a user interface (e.g. GUI 205), such as graphically, by message, and so on. In other embodiments, the best sleep cause suggestions may be provided audibly via speaker 114 or speaker 124 of earphones 100.

Referring again to FIG. 11, operation 1104 includes creating and updating a sleep debt based on the preferred sleep duration and the actual sleep duration. The sleep debt, in one embodiment, is created based on the difference between the preferred sleep duration and the actual sleep duration. Similarly, the sleep debt, in such an embodiment, is updated after each night based on the difference between the preferred sleep duration and the actual sleep duration. The sleep debt may be compared to a sleep debt threshold to determine whether the sleep debt is greater than, less than, or equal to the sleep debt threshold. In one embodiment, the best sleep cause suggestions are provided when the sleep debt exceeds the sleep debt threshold. For example, if the sleep debt threshold is two hours, and the sleep debt exceeds two hours, it may be suggested that the user exercise in the morning (if morning exercise has been determined to be a best sleep cause).

In one instance, the sleep debt is updated periodically. For example, the sleep debt may represent the average of the difference between the preferred sleep duration and the actual sleep duration over a period of ten days. To illustrate, the sleep debt may reflect that the user is, on average, twenty minutes behind per night over the last ten days. This would mean that, on average, the actual sleep duration measured was twenty minutes less than the preferred sleep duration. The sleep debt may also be negative, indicating that the actual sleep duration was greater than the preferred sleep duration.

Referring again to FIG. 11, operation 1106 involves providing a recommended sleep duration based on the sleep debt (determined at operation 1104). In one embodiment, the recommended sleep duration aids the user in eliminating the user's sleep debt. For example, if the user has a sleep debt of twenty minutes per night averaged over the last ten days, the recommended sleep duration may be that the user get forty minutes more than the user's preferred sleep over the next five days. This may help the user to approximate the user's preferred sleep duration in the long run and overcome the user's sleep debt. In one embodiment, the recommended sleep duration helps the user to gradually eliminate the user's sleep debt. This may be more beneficial than, for example, attempting to eliminate the sleep debt in a single or just a few nights.

In one embodiment, the recommended sleep duration is further based on a fatigue level. For example, a higher fatigue level may correspond to a longer recommended sleep duration, while a lower fatigue level may correspond to a shorter recommended sleep duration. The fatigue level may be detected in various ways. In one example, the fatigue level is detected by measuring a heart rate variability (HRV) of the user using earphones 100 (discussed above in reference to FIGS. 1-4B). For example, optical heartrate sensor 122 may also be used to estimate a heart rate variable (HRV), i.e. the variation in time interval between consecutive heartbeats, of the user of earphones 100. For example, processor 165 may calculate the HRV using the data collected by sensor 122 based on a time domain methods, frequency domain methods, and other methods known in the art that calculate HRV based on data such as the mean heart rate, the change in pulse rate over a time interval, and other data used in the art to estimate HRV. Further, possible representations of the fatigue level are described above (e.g., numerical, descriptive, etc.). When the HRV is more consistent (i.e., steady, consistent amount of time between heartbeats), for example, the fatigue level may be higher. When HRV is more sporadic (i.e., amount of time between heartbeats varies largely), the fatigue level may be lower. In general, with a lower fatigue level, the body is more fresh and well-rested.

HRV may be measured in a number of ways. Measuring HRV, in one embodiment, involves the combination of optical heartrate sensor 122 of earphones 100 and a finger biosensor that may be coupled to earphones 100 or computing device 200 or both. For example, optical heartrate sensor 122 may measure the heartbeat as detected at the tragus of a user's left ear while a finger sensor measures the heartbeat in a finger of the user's right hand. This combination allows the sensors, which in one embodiment are conductive, to measure an electrical potential through the body. Information about the electrical potential provides cardiac information (e.g., HRV, fatigue level, heart rate information, and so on), and such information may be processed. In other embodiments, the HRV is measured using sensors that monitor other parts of the user's body, rather than the finger and ear. For example, the sensors may monitor the ankle, leg, arm, or torso.

The fatigue level, in another embodiment, factors into determining the preferred sleep duration. For example, if a higher fatigue level is detected after a shorter or longer amount of sleep, this may be useful data in determining the user's preferred sleep duration. The preferred sleep duration may be the sleep duration that minimizes the fatigue level detected following that sleep duration.

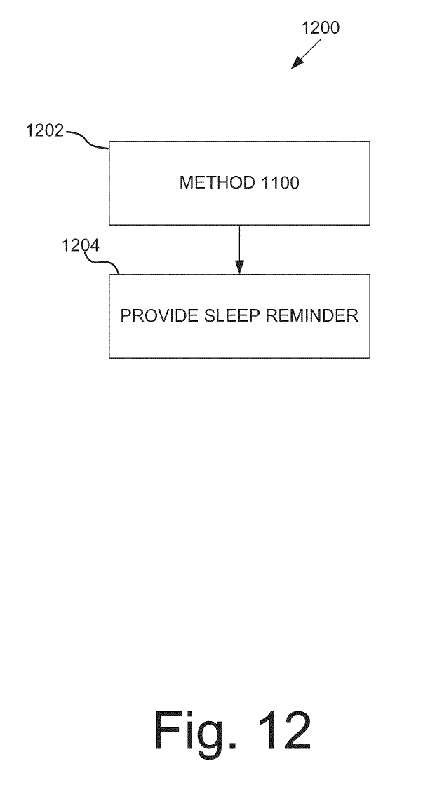

FIG. 12 is an operational flow diagram illustrating example method 1200 for providing a sleep recommendation. In one embodiment, apparatus 802, earphones 100, computing device 808 and/or computing device 200 perform various operations of method 1200. Method 1200, in various embodiments, includes one or more operations of method 1100, represented at operation 1202.

In one embodiment, at operation 1204, method 1200 involves providing a sleep reminder based on the sleep debt. The sleep reminder, in one instance, is provided when the sleep debt exceeds a sleep debt threshold. For example, the sleep debt threshold may be two hours. If the sleep debt exceeds two hours, the sleep reminder may be provided to aid the user in eliminating the sleep debt. In one embodiment, the sleep reminder includes a notification delivered to an electronic device (e.g. computing device 200, computing device 808, etc.), which may include a smartphone, television, tablet, smartwatch, earphones or other device. The notification may be in the form of a text message, a pop-up window, an alert, an audible sound, and so on.

Providing the sleep reminder, in one embodiment, occurs before a preferred bed time of the user. The preferred bed time, similar to the preferred sleep duration, may be based on a combination of user input of an estimated preferred bed time and an empirical preferred bed time based on the user's preferred sleep duration. The empirical preferred bed time, in one embodiment, is the bed time that corresponds to the user's preferred sleep durations. For example, the user may achieve the user's preferred sleep duration when the user goes to bed at a particular time, and the user may accrue significant sleep debt when the user goes to bed at another time (e.g., later at night). The preferred bed time, in one embodiment, updates dynamically in response to changes in the user's empirical preferred sleep durations.

In another embodiment, the user enters the preferred bed time and freezes the preferred bed time, such that the preferred bed time remains fixed, or static. Whether the preferred bed time is fixed or dynamic, the sleep reminder, in one embodiment, is provided before the preferred bed time. The sleep reminder may be provided thirty minutes before the preferred bed time, for example. In one embodiment, this amount of time is programmable by the user. Providing the sleep notification before the preferred bed time may allow the user to get ready for bed and go to sleep at the preferred bed time.

In a further embodiment, the bed time notification is adjusted based on the sleep debt such that the user may comply with the recommended sleep duration—that is, such that the user can get to bed early enough to achieve the recommended sleep duration and still wake up in time to fulfill the user's obligations in the morning. This further aids in eliminating sleep debt. In one case, the bed time notification is synced to one or more calendars, including the user's calendar. This allows for the bed time notification to adjust automatically in anticipation of the user's obligations in the morning and provide the user ample time to eliminate the user's sleep debt.

In various embodiments, at least one of the operations of determining the preferred sleep duration, creating and updating the sleep debt, providing the recommended sleep duration, and providing the sleep reminder includes using a sensor coupled to a processor, both the sensor and the processor being embedded within or coupled to an earphone configured to be attached to the body of the user (e.g. earphones 100).

Returning briefly again to a discussion of the display depicted in FIG. 7, sleep display 700 may comprise a display navigation area 701, a center sleep display area 702, a textual sleep recommendation 703, and a sleeping detail or timeline 704. Display navigation area 701 allows a user to navigate between the various displays associated with modules 211-214 as described above. In this embodiment the sleep display 700 includes the identification “SLEEP” at the center of the navigation area 701.

Center sleep display area 702 may display sleep metrics such as the user's recent average level of sleep or sleep trend 702A, a recommended amount of sleep for the night 702B, and an ideal average sleep amount 702C. In various embodiments, these sleep metrics may be displayed in units of time (e.g., hours and minutes) or other suitable units. Accordingly, a user may compare a recommended sleep level for the user (e.g., metric 702B) against the user's historical sleep level (e.g., metric 702A). In one embodiment, the sleep metrics 702A-702C may be displayed as a pie chart showing the recommended and historical sleep times in different colors. In another embodiment, sleep metrics 702A-702C may be displayed as a curvilinear graph showing the recommended and historical sleep times as different colored, concentric lines. This particular embodiment is illustrated in example sleep display 700, which illustrates an inner concentric line for recommended sleep metric 702B and an outer concentric line for average sleep metric 702A. In this example, the lines are concentric about a numerical display of the sleep metrics.

In various embodiments, a textual sleep recommendation 703 may be displayed at the bottom or other location of display 700 based on the user's recent sleep history. A sleeping detail or timeline 704 may also be displayed as a collapsed bar at the bottom of sleep display 700. In various embodiments, when a user selects sleeping detail 704, it may display a more detailed breakdown of daily sleep metrics, including, for example, total time slept, bedtime, and wake time. In particular implementations of these embodiments, the user may edit the calculated bedtime and wake time. In additional embodiments, the selected sleeping detail 704 may graphically display a timeline of the user's movements during the sleep hours, thereby providing an indication of how restless or restful the user's sleep is during different times, as well as the user's sleep cycles. For the example, the user's movements may be displayed as a histogram plot charting the frequency and/or intensity of movement during different sleep times.

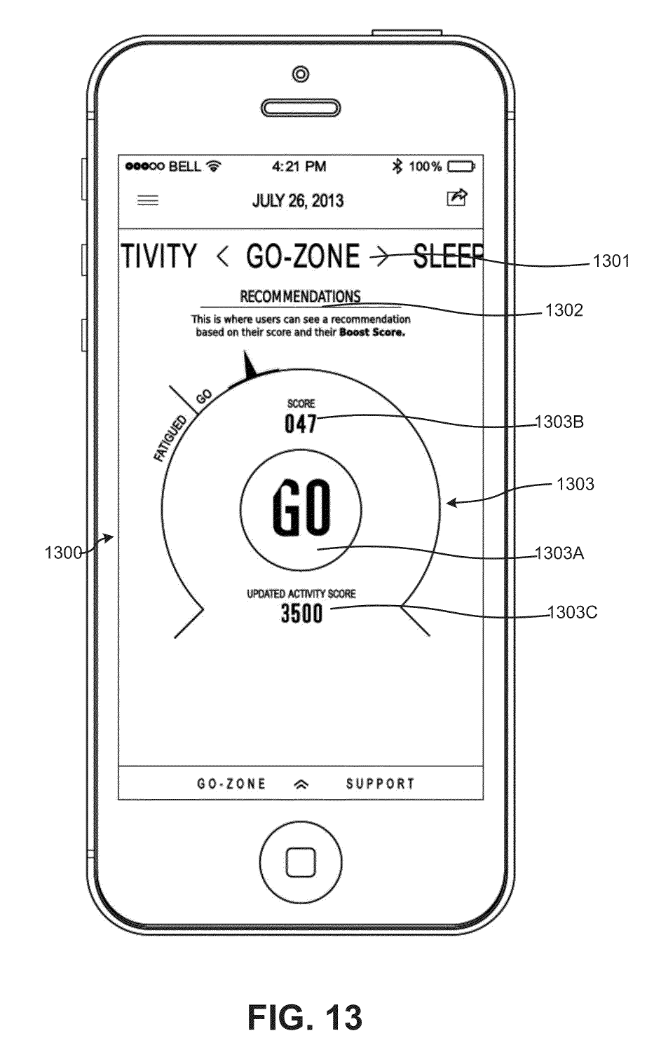

Looking now at further exemplary displays that may be used to implement embodiments of the disclosed technology, FIG. 13 illustrates an activity recommendation and fatigue level display 1300 that may be associated with an activity recommendation and fatigue level display module 213. In various embodiments, display 1300 may visually present to a user the user's current fatigue level and a recommendation of whether or not engage in activity. It is worth noting that one or more modules of activity tracking application 210 may track fatigue level based on data received from the earphones 100, and make an activity level recommendation. For example, HRV data tracked at regular intervals may be compared with other biometric or biological data to determine how fatigued the user is. Additionally, the HRV data may be compared to pre-loaded or learned fatigue level profiles, as well as a user's specified activity goals. Particular systems and methods for implementing this functionality are described in greater detail in U.S. patent application Ser. No. 14/140,414, filed Dec. 24, 2013, titled “System and Method for Providing an Intelligent Goal Recommendation for Activity Level”, and which is incorporated herein by reference in its entirety.

As illustrated, display 1300 may comprise a display navigation area 1301 (as described above), a textual activity recommendation 1302, and a center fatigue and activity recommendation display 1303. Textual activity recommendation 1302 may, for example, display a recommendation as to whether a user is too fatigued for activity, and thus must rest, or if the user should be active. Center display 1303 may display an indication to a user to be active (or rest) 1303A (e.g., “go”), an overall score 1303B indicating the body's overall readiness for activity, and an activity goal score 1303C indicating an activity goal for the day or other period. In various embodiments, indication 1303A may be displayed as a result of a binary decision—for example, telling the user to be active, or “go”—or on a scaled indicator—for example, a circular dial display showing that a user should be more or less active depending on where a virtual needle is pointing on the dial.

In various embodiments, display 1300 may be generated by measuring the user's HRV at the beginning of the day (e.g., within 30 minutes of waking up.) For example, the user's HRV may be automatically measured using the optical heartrate sensor 122 after the user wears the earphones in a position that generates a good signal as described in method 500. In embodiments, when the user's HRV is being measured, computing device 200 may display any one of the following: an instruction to remain relaxed while the variability in the user's heart signal (i.e., HRV) is being measured, an amount of time remaining until the HRV has been sufficiently measured, and an indication that the user's HRV is detected. After the user's HRV is measured by earphones 100 for a predetermined amount of time (e.g., two minutes), one or more processing modules of computing device 200 may determine the user's fatigue level for the day and a recommended amount of activity for the day. Activity recommendation and fatigue level display 1300 is generated based on this determination.

In further embodiments, the user's HRV may be automatically measured at predetermined intervals throughout the day using optical heartrate sensor 122. In such embodiments, activity recommendation and fatigue level display 1300 may be updated based on the updated HRV received throughout the day. In this manner, the activity recommendations presented to the user may be adjusted throughout the day.

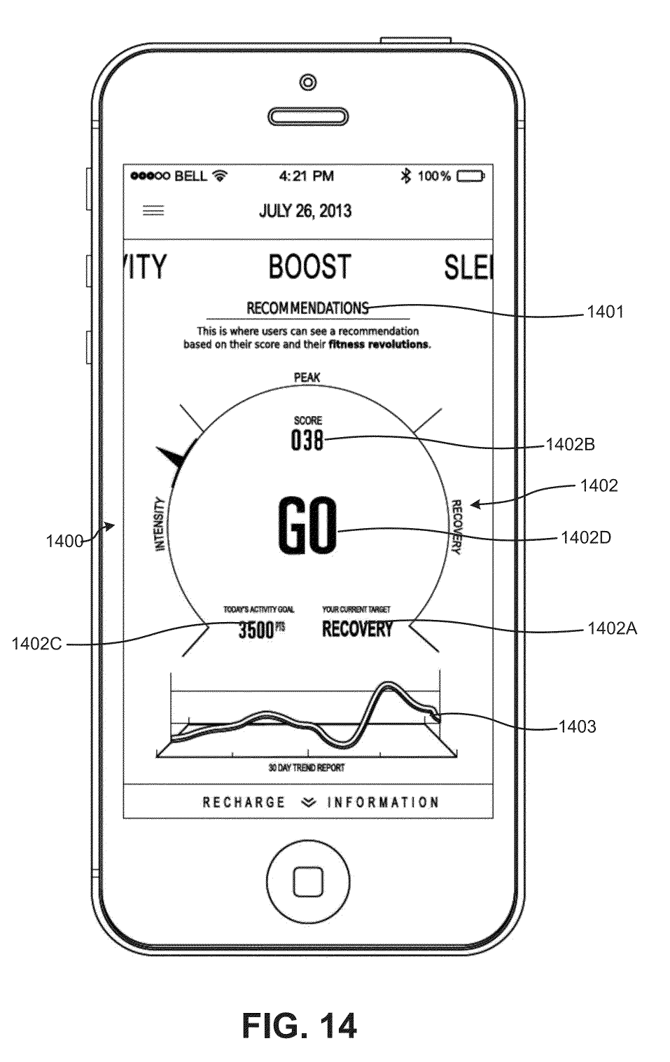

FIG. 14 illustrates a biological data and intensity recommendation display 1400 that may be associated with a biological data and intensity recommendation display module 214. In various embodiments, display 1400 may guide a user of the activity monitoring system through various fitness cycles of high-intensity activity followed by lower-intensity recovery based on the user's body fatigue and recovery level, thereby boosting the user's level of fitness and capacity on each cycle.

As illustrated, display 1400 may include a textual recommendation 1401, a center display 1402, and a historical plot 1403 indicating the user's transition between various fitness cycles. In various embodiments, textual recommendation 1401 may display a current recommended level of activity or training intensity based on current fatigue levels, current activity levels, user goals, pre-loaded profiles, activity scores, smart activity scores, historical trends, and other bio-metrics of interest. Center display 1402 may display a fitness cycle target 1402A (e.g., intensity, peak, fatigue, or recovery), an overall score 1402B indicating the body's overall readiness for activity, an activity goal score 1402C indicating an activity goal for the day or other period, and an indication to a user to be active (or rest) 1402D (e.g., “go”). The data of center display 1402 may be displayed, for example, on a virtual dial, as text, or some combination thereof. In one particular embodiment implementing a dial display, recommended transitions between various fitness cycles (e.g., intensity and recovery) may be indicated by the dial transitioning between predetermined markers.

In various embodiments, display 1400 may display a historical plot 1403 that indicates the user's historical and current transitions between various fitness cycles over a predetermined period of time (e.g., 30 days). The fitness cycles, may include, for example, a fatigue cycle, a performance cycle, and a recovery cycle. Each of these cycles may be associated with a predetermined score range (e.g., overall score 1402B). For example, in one particular implementation a fatigue cycle may be associated with an overall score range of 0 to 33, a performance cycle may be associated with an overall score range of 34 to 66, and a recovery cycle may be associated with an overall score range of 67 to 100. The transitions between the fitness cycles may be demarcated by horizontal lines intersecting the historical plot 1403 at the overall score range boundaries. For example, the illustrated historical plot 1403 includes two horizontal lines intersecting the historical plot. In this example, measurements below the lowest horizontal line indicate a first fitness cycle (e.g., fatigue cycle), measurements between the two horizontal lines indicate a second fitness cycle (e.g., performance cycle), and measurements above the highest horizontal line indicate a third fitness cycle (e.g., recovery cycle).

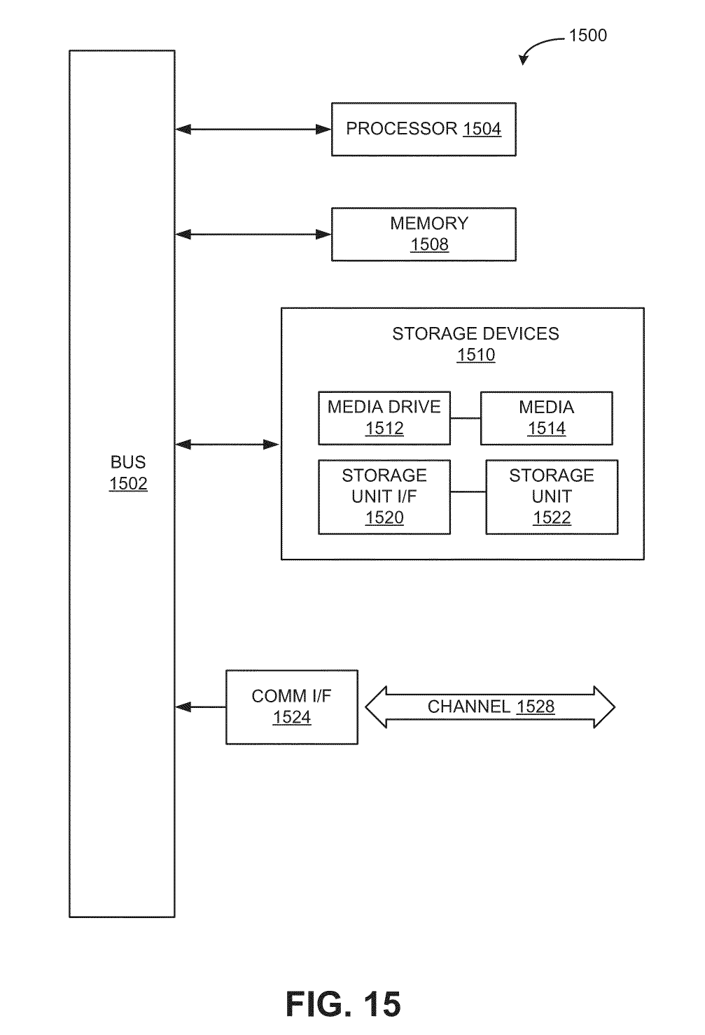

FIG. 15 illustrates an example computing module that may be used to implement various features of the systems and methods disclosed herein. In one embodiment, the computing module includes a processor and a set of computer programs residing on the processor. The set of computer programs is stored on a non-transitory computer readable medium having computer executable program code embodied thereon. The computer executable code is configured to determine a preferred sleep duration. The computer executable code is further configured to create and update a sleep debt based on the preferred sleep duration and an actual sleep duration. In addition, the computer executable code is configured to provide a recommended sleep duration based on the sleep debt.

The example computing module may be used to implement these various features in a variety of ways, as described above with reference to the methods illustrated in FIGS. 10 and 11 and as will be appreciated by one of ordinary skill in the art.

As used herein, the term module might describe a given unit of functionality that can be performed in accordance with one or more embodiments of the present application. As used herein, a module might be implemented utilizing any form of hardware, software, or a combination thereof. For example, one or more processors, controllers, ASICs, PLAs, PALs, CPLDs, FPGAs, logical components, software routines or other mechanisms might be implemented to make up a module. In implementation, the various modules described herein might be implemented as discrete modules or the functions and features described can be shared in part or in total among one or more modules. In other words, as would be apparent to one of ordinary skill in the art after reading this description, the various features and functionality described herein may be implemented in any given application and can be implemented in one or more separate or shared modules in various combinations and permutations. Even though various features or elements of functionality may be individually described or claimed as separate modules, one of ordinary skill in the art will understand that these features and functionality can be shared among one or more common software and hardware elements, and such description shall not require or imply that separate hardware or software components are used to implement such features or functionality.

Where components or modules of the application are implemented in whole or in part using software, in one embodiment, these software elements can be implemented to operate with a computing or processing module capable of carrying out the functionality described with respect thereto. One such example computing module is shown in FIG. 15. Various embodiments are described in terms of this example-computing module 1500. After reading this description, it will become apparent to a person skilled in the relevant art how to implement the application using other computing modules or architectures.

Referring now to FIG. 15, computing module 1500 may represent, for example, computing or processing capabilities found within desktop, laptop, notebook, and tablet computers; hand-held computing devices (tablets, PDA's, smart phones, cell phones, palmtops, smart-watches, smart-glasses etc.); mainframes, supercomputers, workstations or servers; or any other type of special-purpose or general-purpose computing devices as may be desirable or appropriate for a given application or environment. Computing module 1500 might also represent computing capabilities embedded within or otherwise available to a given device. For example, a computing module might be found in other electronic devices such as, for example, digital cameras, navigation systems, cellular telephones, portable computing devices, modems, routers, WAPs, terminals and other electronic devices that might include some form of processing capability.

Computing module 1500 might include, for example, one or more processors, controllers, control modules, or other processing devices, such as a processor 1504. Processor 1504 might be implemented using a general-purpose or special-purpose processing engine such as, for example, a microprocessor, controller, or other control logic. In the illustrated example, processor 1504 is connected to a bus 1502, although any communication medium can be used to facilitate interaction with other components of computing module 1500 or to communicate externally.

Computing module 1500 might also include one or more memory modules, simply referred to herein as main memory 1508. For example, preferably random access memory (RAM) or other dynamic memory, might be used for storing information and instructions to be executed by processor 1504. Main memory 1508 might also be used for storing temporary variables or other intermediate information during execution of instructions to be executed by processor 1504. Computing module 1500 might likewise include a read only memory (“ROM”) or other static storage device coupled to bus 1502 for storing static information and instructions for processor 1504.

The computing module 1500 might also include one or more various forms of information storage mechanism 1510, which might include, for example, a media drive 1512 and a storage unit interface 1520. The media drive 1512 might include a drive or other mechanism to support fixed or removable storage media 1514. For example, a hard disk drive, a solid state drive, a magnetic tape drive, an optical disk drive, a CD or DVD drive (R or RW), or other removable or fixed media drive might be provided. Accordingly, storage media 1514 might include, for example, a hard disk, a solid state drive, magnetic tape, cartridge, optical disk, a CD or DVD, or other fixed or removable medium that is read by, written to or accessed by media drive 1512. As these examples illustrate, the storage media 1514 can include a computer usable storage medium having stored therein computer software or data.

In alternative embodiments, information storage mechanism 1510 might include other similar instrumentalities for allowing computer programs or other instructions or data to be loaded into computing module 1500. Such instrumentalities might include, for example, a fixed or removable storage unit 1522 and a storage interface 1520. Examples of such storage units 1522 and storage interfaces 1520 can include a program cartridge and cartridge interface, a removable memory (for example, a flash memory or other removable memory module) and memory slot, a PCMCIA slot and card, and other fixed or removable storage units 1522 and storage interfaces 1520 that allow software and data to be transferred from the storage unit 1522 to computing module 1500.

Computing module 1500 might also include communications interface 1524. Communications interface 1524 might be used to allow software and data to be transferred between computing module 1500 and external devices. Examples of communications interface 1524 might include a modem or softmodem, a network interface (such as an Ethernet, network interface card, WiMedia, IEEE 902.XX or other interface), a communications port (such as for example, a USB port, IR port, RS232 port Bluetooth® interface, or other port), or other communications interface. Software and data transferred via communications interface 1524 might typically be carried on signals, which can be electronic, electromagnetic (which includes optical) or other signals capable of being exchanged by a given communications interface 1524. These signals might be provided to communications interface 1524 via a channel 1528. This channel 1528 might carry signals and might be implemented using a wired or wireless communication medium. Some examples of a channel might include a phone line, a cellular link, an RF link, an optical link, a network interface, a local or wide area network, and other wired or wireless communications channels.

In this document, the terms “computer program medium” and “computer usable medium” are used to generally refer to transitory or non-transitory media such as, for example, memory 1508, storage unit 1520, media 1514, and channel 1528. These and other various forms of computer program media or computer usable media may be involved in carrying one or more sequences of one or more instructions to a processing device for execution. Such instructions embodied on the medium are generally referred to as “computer program code” or a “computer program product” (which may be grouped in the form of computer programs or other groupings). When executed, such instructions might enable computing module 1500 to perform features or functions of the present application as discussed herein.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

While various embodiments of the present disclosure have been described above, it should be understood that these embodiments have been presented by way of example only, and not of limitation. Likewise, the various diagrams may depict an example architectural or other configuration for the disclosure, which is done to aid in understanding the features and functionality that can be included in the disclosure. The disclosure is not restricted to the illustrated example architectures or configurations, but the desired features can be implemented using a variety of alternative architectures and configurations. Indeed, it will be apparent to one of skill in the art how alternative functional, logical, or physical partitioning and configurations can be implemented to implement the desired features of the present disclosure. Also, a multitude of different constituent module names other than those depicted herein can be applied to the various partitions. Additionally, with regard to flow diagrams, operational descriptions, and method claims, the order in which the steps are presented herein does not mandate that various embodiments be implemented to perform the recited functionality in the same order, unless the context dictates otherwise.

Although the disclosure is described above in terms of various exemplary embodiments and implementations, it should be understood that the various features, aspects, and functionalities described in one or more of the individual embodiments are not limited in their applicability to the particular embodiment with which they are described, but instead can be applied, alone or in various combinations, to one or more of the other embodiments of the disclosure, whether or not such embodiments are described and whether or not such features are presented as being a part of a described embodiment. Thus, the breadth and scope of the present disclosure should not be limited by any of the above-described exemplary embodiments.

What is claimed is:

1. An system for providing a sleep recommendation comprising:
an earphone comprising:
a speaker;
a motion sensor;
a processor communicatively coupled to the motion sensor, wherein the processor is configured to process electronic input signals from the motion sensor; and
a nontransitory computer-readable medium operatively coupled to the processor and having instructions stored thereon that, when executed, cause the system to:
determine a preferred sleep duration;
create and update a sleep debt measure based on the preferred sleep duration and an actual sleep duration; and
provide a recommended sleep duration based on the sleep debt measure.

2. The system of claim 1, wherein the preferred sleep duration is based on a set of best sleep durations for a user.

3. The system of claim 2, wherein the set of best sleep durations is based on a set of the actual sleep durations.

4. The system of claim 1, further comprising a sleep reminder module that provides a sleep reminder based on the sleep debt.

5. The system of claim 4, wherein the instructions, when executed, further cause the system to provide the sleep reminder when the sleep debt exceeds a sleep debt threshold.

6. The system of claim 4, wherein the sleep reminder comprises a notification delivered to an electronic device.

7. The system of claim 4, wherein the instructions, when executed, further cause the system to provide the sleep reminder before a preferred bed time.

8. The system of claim 1, wherein the preferred sleep duration is based on a needed sleep duration provided by a user.

9. The system of claim 1, further comprising an actual sleep determination module that determines the actual sleep duration using an accelerometer.

10. The system of claim 1, wherein at least one of the nontransitory computer-readable medium and the processor is embedded in the earphone.

11. The system of claim 1, further comprising a heartrate sensor electrically coupled to the processor of the earphone; and wherein the processor is configured to process electronic input signals from the motion sensor and the heartrate sensor.

12. The system of claim 11, wherein the heartrate sensor is an optical heartrate sensor.

13. The system of claim 1, wherein at least one of the nontransitory computer-readable medium and the processor resides in a computing device configured to receive data from the earphones through wireless communication.

14. A method for providing a sleep recommendation using an earphone, comprising:
receiving electronic signals generated by a sensor coupled to an earphone;
determining a preferred sleep duration;
determining an actual sleep duration based on the electronic signals received from the sensor;
creating and updating a sleep debt based on the preferred sleep duration and the actual sleep duration; and
providing a recommended sleep duration based on the sleep debt.

15. The method of claim 14, wherein the preferred sleep duration is based on a set of best sleep durations for a user.

16. The method of claim 15, wherein the set of best sleep durations is based on a set of the actual sleep determinations.

17. The method of claim 14, further comprising providing a sleep reminder based on the sleep debt.

18. The method of claim 17, wherein providing the sleep reminder occurs in response to the sleep debt exceeding a sleep debt threshold.

19. The method of claim 17, wherein the sleep reminder comprises a notification delivered to an electronic device.

20. The method of claim 17, wherein the sleep reminder comprises a notification delivered to a smartphone.

21. The method of claim 17, wherein the sleep reminder comprises a notification delivered audibly through a speaker.

22. The method of claim 17, wherein providing the sleep reminder occurs before a preferred bed time.

23. The method of claim 14, wherein the sensor is an accelerometer.

24. The method of claim 14, wherein the sensor is an optical heartrate sensor.

25. The method of claim 14, wherein at least one of the operations of determining the preferred sleep duration, creating and updating the sleep debt, and providing the recommended sleep duration comprises using a sensor coupled to an earphone.

26. A system for providing a sleep recommendation, comprising:
one or more processors; and
at least one computer program residing on one of the one or more processors;
wherein the computer program is stored on a non-transitory computer readable medium having computer executable program code embodied thereon, the computer executable program code configured to:
determine a preferred sleep duration;
create and update a sleep debt based on the preferred sleep duration and an actual sleep duration; and
provide a recommended sleep duration based on the sleep debt
a display configured to display the recommended sleep duration.

27. The system of claim 26, wherein the actual sleep duration is determined using data received from a sensor coupled to an earphone.

28. The system of claim 27, wherein the sensor is an accelerometer.

29. The system of claim 27, wherein the sensor is an optical heartrate sensor.

30. The system of claim 27, wherein the sensor is an optical heartrate sensor protruding from a side of the earphone proximal to an interior side of a user's ear when the earphone is worn, and wherein the optical heartrate sensor is configured to measure the user's blood oxygenation level and to output an electrical signal representative of this measurement to one of the one or more processors.
